(19) United States
(12) Patent Application Publication
(10) Pub. No.: US 2023/0212593 A1
(43) Pub. Date: Jul. 6, 2023

(54) METHOD FOR THE PRODUCTION OF CONSTITUTIVE BACTERIAL PROMOTERS CONFERRING LOW TO MEDIUM EXPRESSION

(71) Applicant: BASF SE, Ludwigshafen Am Rhein (DE)

(72) Inventors: Max Fabian FELLE, Ludwigshafen (DE); Christopher SAUER, Ludwigshafen (DE); Norma WELSCH, Greifswald (DE); Mathis APPELBAUM, Ludwigshafen (DE); Thomas SCHWEDER, Greifswald (DE)

(21) Appl. No.: 17/905,499

(22) PCT Filed: Mar. 1, 2021
(86) PCT No.: PCT/EP2021/054993

§ 371 Date: Sep. 1, 2022

(30) Foreign Application Priority Data
Mar. 4, 2020 (EP) 20160961.7

(51) Int. Cl. C12N 15/69; C12N 15/10; C12N 15/75

(52) U.S. Cl. CPC C12N 15/69; C12N 15/1034; C12N 15/75; C12N 2310/20; C12N 2820/55

(57) ABSTRACT

Disclosed herein are methods for the production of low to medium expressing constitutive promoters in bacteria and promoters produced therewith.

### CROSS-REFERENCE TO RELATED APPLICATIONS

This application is a U.S. National Phase Application of International Patent Application No. PCT/EP2021/054993, filed Mar. 1, 2021, which claims priority to European Patent Application No. 20160961.7, filed Mar. 4, 2020, each of which is hereby incorporated by reference herein.

### REFERENCE TO AN ELECTRONIC SEQUENCE LISTING

The contents of the electronic sequence listing (27843-1997_ST25.txt; Size: 48,459 bytes; and Date of Creation: Jun. 28, 2022) are herein incorporated by reference in their entirety.

### DESCRIPTION OF THE INVENTION

The present invention is in the field of molecular biology and provides methods for the production of low to medium expressing constitutive promoters in bacteria and promoters produced therewith.

### INTRODUCTION

Microorganisms are nowadays widely applied in industry by making use of their fermentation capacity. Microorganisms are particularly used as a host for fermentative production of a variety of substances such as enzymes, proteins, chemicals, sugars and polymers. For these purposes, microorganisms are subject of genetic engineering in order to adapt their gene expression to the demands of the specific production process. Rational genetic engineering of microorganism requires technologies for target specific genome editing such as introduction of point mutations, gene deletion, gene insertions, gene duplications.

Many different approaches for genome editing for several species have been developed. Most of them require introduction of a double strand DNA break or two adjacent single stand DNA breaks to introduce random mutations at a specific site in the genome by nonhomologous end-joining (NHEJ) or to introduce, replace or delete DNA using a homologous recombination repair mechanism (HR) which requires delivery of a donor DNA molecule. Technologies used were for example Zn-finger nucleases, TALENs, homing endonucleases and the like. The recent development of CRISPR (clustered regularly interspaced short palindromic repeats) based systems made genome editing even more attractive, due to its precision efficiency and speed.

The CRISPR system was initially identified as an adaptive defense mechanism of bacteria belonging to the genus of Streptococcus (WO2007/025097). Those bacterial CRISPR systems rely on guide RNA (gRNA) in complex with cleaving proteins to direct degradation of complementary sequences present within invading viral DNA. Cas9, the first identified protein of the CRISPR/Cas system, is a large monomeric DNA nuclease guided to a DNA target sequence adjacent to the PAM (protospacer adjacent motif) sequence motif by a complex of two noncoding RNAs: crRNA and trans-activating crRNA (tracrRNA). Later, a synthetic RNA chimera (single guide RNA or sgRNA) created by fusing crRNA with tracrRNA was shown to be equally functional (Jinek, M., Chylinski, K., Fonfara, I., Hauer, M., Doudna, J. A., and Charpentier, E. A programmable dual-RNA-guided DNA endonuclease in adaptive bacterial immunity. Science 337(6096), 816-821. 17-8-2012).

Several research groups have found that the CRISPR cutting properties could be used to disrupt genes in almost any organism's genome with unprecedented ease (Mali P, et al (2013) Science. 339(6121):819-823; Cong L, et al (2013) Science 339(6121)). Recently it became clear that providing a template for repair allowed for editing the genome with nearly any desired sequence at nearly any site, transforming CRISPR into a powerful gene editing tool (WO/2014/150624, WO/2014/204728).

A key element to drive gene expression in a host cell is the promoter sequence. For gene expression to take place, the RNA polymerase must attach to the promoter sequence near a gene. Thus, promoters contain specific DNA sequences that provide a binding site for RNA polymerase and also for other proteins that recruit RNA polymerase to the recognition sequence (i.e., transcription factors). In bacteria, the promoter is usually recognized by the RNA polymerase and an associated sigma factor, which are guided to the promoter DNA by an activator protein's binding to its own DNA binding site nearby (Lee, D. J., Minchin, S. D., and Busby, S. J. Activating transcription in bacteria. Annu. Rev. Microbiol. 66, 125-152. 2012.). Constitutive promoters for example driving expression of many house-keeping genes, are independent of activation or derepression by activator or repressor proteins and RNA polymerase binds to the constitutive promoter through the associated sigma factor sigA (also referred to sig70 in E. coli) which recognizes sigA-specific DNA sequence elements −35 box and −10 box. The sigA dependent promoters have been well studied for Bacillus and E. coli and comparison of consensus motifs of sigA promoter sequences indicates cross-recognition of Bacillus and E. coli derived sigA promoters by E. coli and Bacillus RNA-Polymerase with corresponding sig70 and sigA factors respectively (Heimann, J. D. Compilation and analysis of Bacillus subtilis sigma A-dependent promoter sequences: evidence for extended contact between RNA polymerase and upstream promoter DNA. Nucleic Acids Res. 23(13), 2351-2360. 11-7-1995.).

In eukaryotes, the process is more complicated, and various factors are necessary for the binding of an RNA polymerase to the promoter. Influenced by the nucleic acid sequence, promoters can confer low, moderate or high expression levels and can be constitutive or inducible.

Many constitutive promoters have been described for Bacillus. The promoter Pveg of the veg gene is a well described strong constitutive promoter. Moreover, libraries of expression modules comprising constitutive promoters of Bacillus with different promoter strength have been constructed (Guiziou, S., et al (2016). Nucleic Acids Res. 44(15), 7495-7508).

Inducible promoters are either activated or derepressed by the addition of an inducer molecule to the cells. Thereby, an activation protein binds to a sequence next to the promoter sequence and actively recruits RNA polymerase and associated sigma factor to allow initiation of transcription. Well known described examples are the P_{BAD} promoter from E. coli regulated by the araC that alters its conformation and binds as dimer to the operator sites I₁ and I₂.upon addition of arabinose, and the mannose-inducible promoter system PmanP from Bacillus regulated by the activator manR. Inducible promoters such as lacUV5 promoter, the T7-phage promoter for expression in E. coli and the Pspac-I and Ppac-I promoters in Bacillus are negatively regulated by the lac repressor (encoded by lacl gene) binding in the absence of an inducer molecule to its specific lac operator sites either within the promoter sequences, e.g. between the −35 and −10 sigA recognition sites, or vicinity, i.e 3′ or 5′ of the promoter sequence to prevent transcription. Another example is the PxylA inducible promoter system from Bacillus megaterium widely used for Bacillus expression systems. The PxylA promoter is negatively regulated by the xylR repressor protein binding comprising the xylR operator sites 3′ of the transcriptional start site.

Inducible promoter systems are generally favorable for cloning in expression vectors as expression of genes under control of such promoters is greatly reduced and therefore the negative impact on e.g. depriving cellular resources, interfering with cellular metabolism and the like minimized, however, tuning of the desired protein expression needs to be carefully analyzed in regards to the amount of inducer molecule added and the timepoint of induction of expression for each strain it is used in. On the contrary, constitutive promoters have the advantage of inducer-independent application not requiring specific regulators or transporters, thereby being active in a wide range of bacteria.

Plasmids are extrachromosomal circular DNA that are autonomously replicating in the host cell, hence independent of the replication of the hosts chromosome.

For autonomous replication, the plasmid comprises an origin of replication enabling the vector to replicate autonomously in the host cell in question. Examples of bacterial origins of replication are the origins of replication of plasmids pUB110, pE194, pC194, pTB19, pAMβ1, pTA1060 permitting replication in Bacillus and plasmids pBR322, colE1, pUC19, pSC101, pACYC177, and pACYC184 permitting replication in E. coli (Sambrook, J. and Russell, D. W. Molecular cloning. A laboratory manual, 3rd ed, Cold Spring Harbor Laboratory Press, Cold Spring Harbor, N.Y. 2001.).

The copy number of a plasmid is defined as the average number of plasmids per bacterial cell or per chromosome under normal growth conditions. Moreover, there exist different types of replication origins (also referred to as replicons) that result in different copy numbers in the bacterial host.

The plasmid replicon pBS72 and the plasmids pTB19 and derivatives pTB51, pTB52 confer low copy number with 6 copies and 1 to 8 copies respectively within Bacillus cells whereas plasmids pE194 and pUB110 confer low-medium copy number with 14-20 and medium copy number with 30-50 copies per cell respectively. Plasmid pE194 was analyzed in more detail (Villafane, et al (1987): J. Bacteriol. 169(10), 4822-4829) and several pE194—cop mutants described having high copy numbers within Bacillus ranging from 85 copies to 202 copies. Moreover, plasmid pE194 is temperature sensitive with stable copy number up to 37° C., however abolished replication above 43° C. In addition, it exists a pE194 variant referred to as pE194ts with 2 point mutations within the replicon region leading to a more drastic temperature sensitivity—stable copy number up to 32° C., however only 1 to 2 copies per cell at 37° C.

In E. coli the pBR322 plasmid carrying the pMB1 replicon or its close relative, the colicine E1 (colE1) replicon maintain low-medium copy number, namely 15-20 copies in each bacterial cell. Deletion of the rop/rom gene within colE1 and pMB1 plasmid derivatives slightly increase the plasmid copy number to medium copy number of 25-50 within the E. coli cell. The pUC vector series are small, high-copy plasmids with up to 200 copies per E. coli cell derived from mutated pBR322 plasmid devoid of the rop protein. The pUC plasmids are well established cloning vectors due to their small size and high yield in plasmid preparations compared to the above mentioned pBR322 and ColE1 derived vectors.

Alternatively, the p15A replicon present in the pACYC177/184 plasmids confers low-medium copy number with 20 copies per cell and the pSC101 replicon low copy number with 5-10 copies per cell. Plasmids with low to medium copy numbers and encoding a toxic or unfavorable expression construct are usually stably maintained within the cell, however, yield in plasmid preparation is low. For subsequent transformation of bacterial cells—the amount of plasmid DNA becomes limiting compared to plasmid preparations of high-copy plasmids. This is in particular of interest for medium to high throughput applications when performing multiple preparations in parallel.

The combination of plasmid copy number and the choice of promoter used for the expression of a gene determines the overall protein expression level and hence the impact on the cell's viability and plasmid stability.

CRISPR-based expression systems for application in gram positive organisms such as Bacillus species based on the single-plasmid system approach, i.e. comprising the Cas9 endonuclease, the gRNA (e.g. sgRNA or crRNA/tracrRNA), repair homology sequences (donor DNA) on one single E. coli-Bacillus shuttle vector have been successfully applied. Altenbuchner created a series of high copy pUC replicon based CRISPR/Cas9 genome editing E. coli-Bacillus shuttle-plasmids for B. subtilis, combined with inducible promoters PmanP, PxylA and PtetLM for the expression of Cas9 endonuclease (Altenbuchner, (2016): Applied and environmental microbiology 82 (17), 5421-5427). This allows highly efficient plasmid DNA preparation and stable maintenance within the E. coli cloning host. Likewise, a similar approach for the construction of a high-copy pUC-derived CRISPRi-E. coli Bacillus shuttle plasmid for application in Bacillus methanolicus was made. The promoter of B. methanolicus mannitol activator gene mtlR driving expression of the defective Cas9 expression was modified by introduction of the lacO site 3′ of the promoter, hence efficiently blocking transcriptional activity in E. coli with intact lacl (Schultenkämper, et al (2019): Applied microbiology and biotechnology 103 (14), 5879-5889).

Another single-plasmid approach for CRISPR/Cas9 application in B. subtilis used the low to medium copy number replicon p15A to allow successful cloning and stable maintenance of CRISPR/Cas9-based genome editing E. coli-Bacillus shuttle plasmids in E. coli in combination with the use of an inducer-independent promotor for Cas9 expression (PamyQ-amylase promoter from B. amyloliquefaciens). A similar combination of medium-copy pBR322 derived E. coli-Bacillus shuttle vector with the Cas9 under the control of a strong constitutive promoter was applied (Zhou, et al. (2019): International journal of biological macromolecules 122, 329-337).

While low and medium copy backbones reduce the metabolic burden, this is accompanied by a reduced plasmid yield from E. coli and impedes isolation of plasmid DNA at the scale required in many protocols for transformation of difficult to transform Bacillus strains or to apply in high-throughput application. Inducer-dependent promoter systems are not always applicable in a wide range of different microorganism and in addition the amount of inducer-molecule and timepoint of promoter induction needs to be analyzed. Moreover, in comparison to constitutive promoters, an additional promoter activation step by adding the inducer molecule to the cell is required stretching the overall timeframe for the genome editing procedure.

Hence there is a need in the art to provide systems that allow the use of high copy vectors in combination with the use of constitutive promoters to overcome these limitations. One element of such system is the provision of constitutive promoters that confer reduced expression in bacteria which does not or only insignificantly interfere with growth and/or vigour of bacteria.

### DETAILED DESCRIPTION OF THE INVENTION

A first embodiment of the invention comprises a method for the of one or more synthetic regulatory nucleic acid molecule, conferring reduced constitutive expression compared to a respective starting regulatory nucleic acid molecule in a bacterial cell comprising the steps of
a. Identifying at least one starting regulatory nucleic acid molecule conferring constitutive expression in a bacterial cell, and
b. Operably linking said starting regulatory nucleic acid molecule to a coding region encoding a protein heterologous to said starting regulatory nucleic acid molecule, and
c. Introducing the construct comprising said starting regulatory nucleic acid molecule operably linked to a coding region into a vector comprising an origin of replication conferring high copy numbers of said vector within a bacterial cell wherein said construct confers high expression of said coding region wherein high expression of said coding region in a bacterial cell burdens said bacterial cell leading to reduced or abolished growth, and
d. Transforming said vector into bacterial cells, and
e. Growing said transformed bacterial cells to recover single clones, and
f. Isolating single clones exhibiting growth rates comparable to corresponding WT strain not comprising said construct, and
g. Isolating from said clones said construct; and
h. Testing the synthetic regulatory nucleic acid molecule comprised in said construct for functional expression of a coding region operably linked to said synthetic regulatory nucleic acid molecule and optionally
i. Comparing the expression conferred by the synthetic regulatory nucleic acid to the expression conferred by the starting regulatory nucleic acid and optionally
j. Sequencing the respective regulatory nucleic acid molecule comprised in said construct, thereby identifying a synthetic regulatory nucleic acid molecule conferring reduced constitutive expression in a bacterial cell.

Reduced growth means that after incubation on a plate for a certain time period under conditions adequate for the respective bacterium a visible difference in the size of a respective colony is visible between colonies of bacteria comprising a construct as described above and colonies of bacteria not comprising said construct. Colonies of bacteria comprising the construct would exhibit smaller colonies as compared to colonies, not comprising said construct. For example, Escherichia coli bacteria would be incubated 8-16 h at 36-37° C. before comparing differences in colony size.

A coding region burdening a bacterium expressing said coding region under control of a strong constitutive promoter could for example be any coding region encoding for a protein larger than 150 kDa, like for example Cas9 or Cas12a, a coding region inducing DNA strand breaks or mutations, like for example Cas9, Cas12a and any other CRISPR Cas enzyme, homing endonucleases, meganucleases, adenosine deaminases or DNA glycosylases, coding regions encoding enzymes interfering with the bacterial metabolism like for example enzymes involved in production of energy equivalents (ATP) or cofactors like NADP, or coding regions encoding transporter or transmembrane proteins interfering with substrate uptake or detoxification of the bacterial cell.

Constitutive expression in a bacterial cell means that the expression strength derived from the respective promoter is substantially constant under various conditions. In this description, constitutive expression means that the expression derived from one promoter differs by less then factor 10, preferably less than factor 9, preferably less than factor 8, preferably less than factor 7, preferably less than factor 6, preferably less than factor 5, preferably less than factor 4, more preferably less than factor 3, even more preferably less than factor 2 under the following conditions: exponential growth phase, transition phase and stationary phase in rich medium, for example LB medium, in rich medium substituted with sugar, for example sucrose, lactose or glucose, preferably glucose in a concentration of between 0.1% to 0.5%, preferably 0.3% and in minimal salt medium, for example M9 medium supplemented with sugar, for example sucrose, lactose or glucose, preferably glucose in a concentration of between 0.1% to 0.5%, preferably 0.3% under temperature conditions optimal for the respective cell.

To determine if a gene is differentially expressed, its expression is measured across these conditions, at least in triplicate and these values are the tested for differences using the DESeq2 package (Love, M. I., et al., Genome Biology 15(12):550 (2014)), a standard approach in the field. Such analysis will estimate the observed fold change between the conditions as well as the probability of such a difference being due to random chance. Any gene which is more up or and/down regulated than defined above and has a probability below 5% of being due to random chance is considered differentially expressed, hence, not constitutively expressed.

Constitutive promoters are independent of other cellular regulating factors and transcription initiation is dependent on sigma factor A (sigA). The sigA-dependent promoters comprise the sigma factor A specific recognition sites ‘−35’-region and ‘−10’-region.

Preferably, the constitutive promoter sequence is selected from the group comprising promoters Pveg, PlepA, PserA, PymdA, Pfba and derivatives thereof with different strength of gene expression (Guiziou et al, (2016): Nucleic Acids Res. 44(15), 7495-7508), bacteriophage SPO1 promoters P4, P5, P15 (WO15118126), the cryIIIA promoter from Bacillus thuringiensis (WO9425612), and combinations thereof, or active fragments or variants thereof.

An origin of replication (ORI) conferring high copy number means an ORI which leads to at least 51 copies of the respective vector in the respective bacterial cell in which the ORI is functional. As the number of copies depend on the temperature under which the respective bacteria is grown, preferably the definition refers to the temperature under which the respective bacterium is grown in the laboratory known to a skilled person as for example described for various strains (Bronikowski et al, (2001): Evolution 55(1):33-40) Preferably for E. coli this means the copy number detected under growth at 36-37° C., for Bacillus this means the copy number detected under growth at 36-37° C.

An ORI conferring medium copy number means an ORI maintaining 25-50 copies of the vector, an ORI conferring low-medium copy number means an ORI maintaining 11-24 copies per cell and an ORI conferring low copy numbers means an ORI maintaining 1-10 copies of the vector within a bacterial cell.

In a preferred embodiment, the E. coli ORI is selected from high copy number ORIs and the Bacillus ORI is selected from low copy number ORIs, low-medium copy number ORIs and medium copy number ORIs.

More preferably, the E. coli ORI is selected from high copy number ORIs and the Bacillus ORI is selected from low-medium copy number ORIs.

More preferably, the E. coli ORI is selected from high copy number ORIs and the Bacillus ORI is selected from low-medium copy number ORIs being temperature sensitive e.g. derivatives of the plasmid pE194 conferring low-medium copy number at 36-37° C. and low-medium copy number at 30-33° C. and no replication above 43° C.

Most preferably, the E. coli ORI is selected from high copy number ORIs, for example the pUC ORI, and the Bacillus ORI is selected from low-medium copy number ORIs being temperature sensitive e.g. derivatives of the plasmid pE194ts conferring low copy number at 36-37° C. and low-medium copy number at 30-33° C. and no replication above 38° C.

The term “clones exhibiting growth rates comparable to corresponding WT strain not comprising said construct” means clones transformed with the construct as defined above that exhibit a growth rate when compared to a bacterium not comprising or not being transformed with such construct having at least 50% of the growth rate as the WT bacteria. Preferably they have at least 60%, 65%, 70%, 75%, 80%, 85% of the growth rate as the WT bacteria. More preferably the have at least 90%, 95% of the growth rate as the WT bacteria or the have a growth rate identical to the WT bacteria. Growth rate can for example be determined by cell density after a certain time of incubation in liquid culture or by colony size on a plate.

Functional expression of a coding region means that the expression of such coding region is at least detectable for example by RNA detection methods like RT-PCR, qPCR or by using detectable proteins like fluorescence proteins, GUS, enzyme reactions specific for the respective enzyme or gene deletion efficiency for coding regions encoding enzymes inducing double strand breaks in the genome, such as CRISPR/Cas enzymes.

A further embodiment of the invention is the method as defined above, wherein the synthetic regulatory nucleic acid molecule confers low to medium high expression in a bacterial cell distinct from the cell in which the recombinant nucleic acid is produced. For example, the starting regulatory nucleic acid molecule is tested and mutated in E. coli and later used for low to medium constitutive expression in Bacillus species. For this purpose, the construct as used in the method defined above may be cloned into a shuttle vector comprising a high copy ORI for E. coli and another ORI of choice for Bacillus species.

In a further embodiment of the invention, the synthetic regulatory nucleic acid molecule is active in cells of gram-positive and gram-negative bacteria, preferably in cells of the class of Bacilli and of the class of Gammaproteobacteria, more preferably in cells of the family of Bacillaceae and the family of Enterobacteriaceae, even more preferably in cells of the genus Bacilli and the genus Escherichia, even more preferably in cells of the genus Bacilli. Preferred cells of the genus Bacilli comprise cells of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus, Bacillus paralicheniformis, Bacillus subtilis, and Bacillus thuringiensis.

Preferably the synthetic regulatory nucleic acid molecule is active in cells of at least three different Bacilli species, in cells of at least two different Bacilli species or in cells of at least one Bacilli species.

More preferably the Bacilli species comprise at least one of Bacillus subtilis, Bacillus licheniformis or Bacillus pumilus. Most preferably the synthetic regulatory nucleic acid molecule is active in cells of Bacillus licheniformis.

In a further embodiment of the invention, any high expression conferring constitutive regulatory nucleic acid molecule active in bacteria may be used. Guiziou et al (Guiziou et al, (2016): Nucleic Acids Res. 44(15), 7495-7508) describe various regulatory nucleic acid molecules that are suitable for the method of the invention and further introduce methods how to identify additional suitable regulatory nucleic acid molecules for the method of the invention. Preferably the starting regulatory nucleic acid molecule conferring high constitutive expression in a bacterial cell is selected from the group consisting of
a) SEQ ID NO: 28 and 29,
b) a nucleic acid molecule comprising at least 20, preferably 25, more preferably 50, more preferably 75, more preferably 100, even more preferably 110, even more preferably 120 consecutive base pairs identical to 20, preferably 25, more preferably 50, more preferably 75, more preferably 100, even more preferably 110, even more preferably 120 consecutive base pairs of a sequence described by SEQ ID NOs: 28 or 29, and
c) a nucleic acid molecule having an identity of at least 90%, preferably at least 91%, 92%, 93%, 94% or 95%, more preferably at least 96%, 97%, 98% or 99% over the entire length of a sequence described by SEQ ID NO: 28 or 29, and
d) a nucleic acid molecule hybridizing under high stringent conditions with a nucleic acid molecule of at least 20 consecutive base pairs, preferably 25, more preferably 50, more preferably 75, more preferably 100, even more preferably 110, even more preferably 120 of a nucleic acid molecule described by SEQ ID NO: 28 or 29 and
e) a complement of any of the nucleic acid molecules as defined in a) to d).

A further embodiment of the invention is a synthetic regulatory nucleic acid molecule wherein the regulatory nucleic acid molecule is comprised in the group consisting of

A) a nucleic acid molecule having a sequence of SEQ ID NO 35, 36, 37, 38, 39, 40, 42, 43, 45, 46 or 47, and

B) a nucleic acid molecule comprising at least 20, preferably 25, more preferably 50, more preferably 75, more preferably 100, even more preferably 110, even more preferably 120 consecutive base pairs identical to 20, preferably 25, more preferably 50, more preferably 75, more preferably 100, even more preferably 110, even more preferably 120 consecutive base pairs of a sequence described by SEQ ID NO: 35, 36, 37, 38, 39, 40, 42, 43, 45, 46 or 47 and
C) a nucleic acid molecule having an identity of at least 90%, preferably at least 91%, 92%, 93%, 94% or 95%, more preferably at least 96%, 97%, 98% or 99% over the entire length to a sequence described by SEQ ID NO: 35, 36, 37, 38, 39, 40, 42, 43, 45, 46 or 47 and
D) a nucleic acid molecule hybridizing under high stringent conditions with a nucleic acid molecule of at least 20, preferably 25, more preferably 50, more preferably 75, more preferably 100, even more preferably 110, even more preferably 120 consecutive base pairs of a nucleic acid molecule described by any of SEQ ID NO: 35, 36, 37, 38, 39, 40, 42, 43, 45, 46 or 47 and
E) a complement of any of the nucleic acid molecules as defined in A) to D), wherein the sequences as defined in B) to E) are distinct from the respective starting regulatory nucleic acid molecule having SEQ ID NO 28 or 29 and preferably comprising at least one base deletion or insertion compared to the respective starting regulatory nucleic acid.

A further embodiment of the invention is the synthetic regulatory nucleic acid molecule as described above, wherein the nucleic acid molecule was produced applying a method as defined above.

An expression construct comprising a synthetic regulatory nucleic acid molecule as defined above is also an embodiment of the invention. Preferably said expression construct comprises a synthetic regulatory nucleic acid molecule and functionally linked thereto a CRISPR/Cas protein, a meganuclease protein or TALE/N encoding coding region, preferably a CRSIPR/Cas protein which is a Cas9 or Cas12a protein.

A vector comprising a synthetic regulatory nucleic acid molecule as defined above or the expression construct defined above is a further embodiment of the invention.

A further embodiment of the invention is a microorganism comprising a regulatory nucleic acid molecule or the expression construct or the vector as defined above.

### Definitions

Abbreviations: GFP—green fluorescence protein, GUS—beta-Glucuronidase, BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Coding region: As used herein the term “coding region” when used in reference to a structural gene refers to the nucleotide sequences which encode the amino acids found in the nascent polypeptide as a result of translation of a mRNA molecule. The coding region is bounded on the 5′-side by the nucleotide triplet “ATG” which encodes the initiator methionine and on the 3′-side by one of the three triplets which specify stop codons (i.e., TAA, TAG, TGA). Alternatively, the nucleotide triplet can be “GTG” or “TTG” and is recognized as the start nucleotide triplet as 5′ to said nucleotide triplet the ribosome binding site (Shine Dalgarno) is located in a distance of 4 nucleotides to 12 nucleotides. Genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene and the ribosome binding site (Shine Dalgarno) which controls or influences translation of the mRNA. The 3′-flanking region may contain sequences which direct the termination of transcription and post-transcriptional cleavage.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of an untransformed cell.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, to-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In regards to cellular development the promoter can also be specific to a particular stage of development e.g. biofilm formation, sporulation.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements in such a way that each of the regulatory elements can fulfil its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (N.Y.); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

“Gene edit” when used herein means the introduction of a specific mutation at a specific position of the genome of a cell. The gene edit may be introduced by precise editing applying more advanced technologies e.g. using a CRISPR Cas system and a donor DNA, or a CRISPR Cas system linked to mutagenic activity such as a deaminase (WO15133554, WO17070632).

Genome and genomic DNA: The terms “genome” or “genomic DNA” is referring to the heritable genetic information of a host organism. In eukaryotes said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus. In prokaryotes said genomic DNA comprises the chromosomal DNA within the bacterial cell.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT plant, or to which it is operably linked at a different location or position in nature, e.g. in the genome of a WT plant.

Preferably the term “heterologous” with respect to a nucleic acid molecule or DNA, e.g. a NEENA refers to a nucleic acid molecule which is operably linked to, or is manipulated to become operably linked to, a second nucleic acid molecule, e.g. a promoter to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, sepharose beads or any other resin. The hybridisation process can furthermore occur with one of the complementary nucleic acids immobilised to a solid support such as a nitro-cellulose or nylon membrane or immobilised by e.g. photolithography to a carrier, including, but not limited to a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

This formation or melting of hybrids is dependent on various parameters, including but not limited thereto the temperature. An increase in temperature favours melting, while a decrease in temperature favours hybridisation. However, this hybrid forming process is not following an applied change in temperature in a linear fashion: the hybridisation process is dynamic, and already formed nucleotide pairs are supporting the pairing of adjacent nucleotides as well. So, with good approximation, hybridisation is a yes-or-no process, and there is a temperature, which basically defines the border between hybridisation and no hybridisation. This temperature is the melting temperature (Tm). Tm is the temperature in degrees Celsius, at which 50% of all molecules of a given nucleotide sequence are hybridised into a double strand, and 50% are present as single strands.

The melting temperature (Tm) is dependent from the physical properties of the analysed nucleic acid sequence and hence can indicate the relationship between two distinct sequences. However, the melting temperature (Tm) is also influenced by various other parameters, which are not directly related with the sequences, and the applied conditions of the hybridization experiment must be taken into account. For example, an increase of salts (e.g. monovalent cations) is resulting in a higher Tm.

Tm for a given hybridisation condition can be determined by doing a physical hybridisation experiment, but Tm can also be estimated in silico for a given pair of DNA sequences. In this embodiment, the equation of Meinkoth and Wahl (Anal. Biochem., 138:267-284, 1984) is used for stretches having a length of 50 or more bases: Tm=81.5° C.+16.6 (log M)+0.41(% GC)−0.61(% form)−500/L.

M is the molarity of monovalent cations, % GC is the percentage of guanosine and cytosine nucleotides in the DNA stretch, % form is the percentage of formamide in the hybridisation solution, and L is the length of the hybrid in base pairs. The equation is for salt ranges of 0.01 to 0.4 M and % GC in ranges of 30% to 75%.

While above Tm is the temperature for a perfectly matched probe, Tm is reduced by about 1° C. for each 1% of mismatching (Bonner et al., J. Mol. Biol. 81: 123-135, 1973): Tm=[81.5° C.+16.6(log M)+0.41(% GC)−0.61(% formamide)−500/L]−% non-identity.

This equation is useful for probes having 35 or more nucleotides and is widely referenced in scientific method literature (e.g. in: “Recombinant DNA Principles and Methodologies”, James Greene, Chapter “Biochemistry of Nucleic acids”, Paul S. Miller, page 55; 1998, CRC Press), in many patent applications (e.g. in: U.S. Pat. No. 7,026,149), and also in data sheets of commercial companies (e.g. “Equations for Calculating Tm” from www.genomics.agilent.com).

Other formulas for Tm calculations, which are less preferred in this embodiment, might be only used for the indicated cases:

For DNA-RNA hybrids (Casey, J. and Davidson, N. (1977) Nucleic Acids Res., 4:1539):

Tm=79.8° C.+18.5 (log M)+0.58(% GC)+11.8(% GC*% GC)−0.5(% form)−820/L.

For RNA-RNA hybrids (Bodkin, D. K. and Knudson, D. L. (1985) J. Virol. Methods, 10: 45):

Tm=79.8° C.+18.5 (log M)+0.58(% GC)+11.8(% GC*% GC)−0.35(% form)−820/L.

For oligonucleotide probes of less than 20 bases (Wallace, R. B., et al. (1979) Nucleic Acid Res. 6: 3535): Tm=2×n(A+T)+4×n(G+C), with n being the number of respective bases in the probe forming a hybrid.

For oligonucleotide probes of 20-35 nucleotides, a modified Wallace calculation could be applied: Tm=22+1.46 n(A+T)+2.92 n(G+C), with n being the number of respective bases in the probe forming a hybrid.

For other oligonucleotides, the nearest-neighbour model for melting temperature calculation should be used, together with appropriate thermodynamic data:

Tm=(Σ(ΔHd)+ΔHi)/(Σ(ΔSd)+ΔSi+ΔSself+R×ln(cT/b))+16.6 log[Na+]−273.15

(Breslauer, K. J., Frank, R., Blocker, H., Marky, L. A. 1986 Predicting DNA duplex stability from the base sequence. Proc. Natl Acad. Sci. USA 833746-3750; Alejandro Panjkovich, Francisco Melo, 2005. Comparison of different melting temperature calculation methods for short DNA sequences. Bioinformatics, 21 (6): 711-722)

where:

Tm is the melting temperature in degrees Celsius;

Σ(ΔHd) and Σ(ΔSd) are sums of enthalpy and entropy (correspondingly), calculated over all internal nearest-neighbor doublets;

ΔSself is the entropic penalty for self-complementary sequences;

ΔHi and ΔSi are the sums of initiation enthalpies and entropies, respectively;

R is the gas constant (fixed at 1.987 cal/K·mol);

cT is the total strand concentration in molar units;

constant b adopts the value of 4 for non-self-complementary sequences or equal to 1 for duplexes of self-complementary strands or for duplexes when one of the strands is in significant excess.

The thermodynamic calculations assume that the annealing occurs in a buffered solution at pH near 7.0 and that a two-state transition occurs.

Thermodynamic values for the calculation can be obtained from Table 1 in (Alejandro Panjkovich, Francisco Melo, 2005. Comparison of different melting temperature calculation methods for short DNA sequences. Bioinformatics, 21 (6): 711-722), or from the original research papers (Breslauer, K. J., Frank, R., Blocker, H., Marky, L. A. 1986 Predicting DNA duplex stability from the base sequence. Proc. Natl Acad. Sci. USA 833746-3750; SantaLucia, J., Jr, Allawi, H. T., Seneviratne, P. A. 1996 Improved nearest-neighbor parameters for predicting DNA duplex stability. Biochemistry 353555-3562; Sugimoto, N., Nakano, S., Yoneyama, M., Honda, K. 1996 Improved thermodynamic parameters and helix initiation factor to predict stability of DNA duplexes. Nucleic Acids Res. 244501-4505).

For an in silico estimation of Tm according to this embodiment, first a set of bioinformatic sequence alignments between the two sequences are generated. Such alignments can be generated by various tools known to a person skilled in the art, like programs “Blast” (NCBI), “Water” (EMBOSS) or “Matcher” (EMBOSS), which are producing local alignments, or “Needle” (EMBOSS), which is producing global alignments. Those tools should be applied with their default parameter setting, but also with some parameter variations. For example, program “MATCHER” can be applied with various parameter for gapopen/gapextend (like 14/4; 14/2; 14/5; 14/8; 14/10; 20/2; 20/5; 20/8; 20/10; 30/2; 30/5; 30/8; 30/10; 40/2; 40/5; 40/8; 40/10; 10/2; 10/5; 10/8; 10/10; 8/2; 8/5; 8/8; 8/10; 6/2; 6/5; 6/8; 6/10) and program “WATER” can be applied with various parameter for gapopen/gapextend (like 10/0,5; 10/1; 10/2; 10/3; 10/4; 10/6; 15/1; 15/2; 15/3; 15/4; 15/6; 20/1; 20/2; 20/3; 20/4; 20/6; 30/1; 30/2; 30/3; 30/4; 30/6; 45/1; 45/2; 45/3; 45/4; 45/6; 60/1; 60/2; 60/3; 60/4; 60/6), and also these programs shall be applied by using both nucleotide sequences as given, but also with one of the sequences in its reverse complement form. For example, BlastN (NCBI) can be applied with an increased e-value cut-off (e.g. e+1 or even e+10) to also identify very short alignments, especially in data bases of small sizes.

Important is that local alignments are considered, since hybridisation may not necessarily occur over the complete length of the two sequences, but may be best at distinct regions, which then are determining the actual melting temperature. Therefore, from all created alignments, the alignment length, the alignment % GC content (in a more accurate manner, the % GC content of the bases which are matching within the alignment), and the alignment identity has to be determined. Then the predicted melting temperature (Tm) for each alignment has to be calculated. The highest calculated Tm is used to predict the actual melting temperature.

The term “hybridisation over the complete sequence of the invention” as defined herein means that for sequences longer than 300 bases when the sequence of the invention is fragmented into pieces of about 300 to 500 bases length, every fragment must hybridise.

For example, a DNA can be fragmented into pieces by using one or a combination of restriction enzymes. A bioinformatic in silico calculation of Tm is then performed by the same procedure as described above, just done for every fragment. The physical hybridisation of individual fragments can be analysed by standard Southern analysis, or comparable methods, which are known to a person skilled in the art.

The term “stringency” as defined herein is describing the ease by which hybrid formation between two nucleotide sequences can take place. Conditions of a “higher stringency” require more bases of one sequence to be paired with the other sequence (the melting temperature Tm is lowered in conditions of “higher stringency”), conditions of “lower stringency” allow some more bases to be unpaired. Hence the degree of relationship between two sequences can be estimated by the actual stringency conditions at which they are still able to form hybrids. An increase in stringency can be achieved by keeping the experimental hybridisation temperature constant and lowering the salts concentrations, or by keeping the salts constant and increasing the experimental hybridisation temperature, or a combination of these parameter. Also an increase of formamide will increase the stringency. The skilled artisan is aware of additional parameters which may be altered during hybridisation and which will either maintain or change the stringency conditions (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical hybridisation experiment is done by an initial hybridisation step, which is followed by one to several washing steps. The solutions used for these steps may contain additional components, which are preventing the degradation of the analyzed sequences and/or prevent unspecific background binding of the probe, like EDTA, SDS, fragmented sperm DNA or similar reagents, which are known to a person skilled in the art (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical probe for a hybridisation experiment is generated by the random-primed-labelling method, which was initially developed by Feinberg and Vogelstein (Anal. Biochem., 132 (1), 6-13 (1983); Anal. Biochem., 137 (1), 266-7 (1984) and is based on the hybridisation of a mixture of all possible hexanucleotides to the DNA to be labelled. The labelled probe product will actually be a collection of fragments of variable length, typically ranging in sizes of 100-1000 nucleotides in length, with the highest fragment concentration typically around 200 to 400 bp. The actual size range of the probe fragments, which are finally used as probes for the hybridisation experiment, can also be influenced by the used labelling method parameter, subsequent purification of the generated probe (e.g. agarose gel), and the size of the used template DNA which is used for labelling (large templates can e.g. be restriction digested using a 4 bp cutter, e.g. Haelll, prior labeling).

For the present invention, the sequence described herein is analysed by a hybridisation experiment, in which the probe is generated from the other sequence, and this probe is generated by a standard random-primed-labelling method. For the present invention, the probe is consisting of a set of labelled oligonucleotides having sizes of about 200-400 nucleotides. A hybridisation between the sequence of this invention and the other sequence means, that hybridisation of the probe occurs over the complete sequence of this invention, as defined above. The hybridisation experiment is done by achieving the highest stringency by the stringency of the final wash step. The final wash step has stringency conditions comparable to the stringency conditions of at least Wash condition 1: 1.06×SSC, 0.1% SDS, 0% formamide at 50° C., in another embodiment of at least Wash condition 2: 1.06×SSC, 0.1% SDS, 0% formamide at 55° C., in another embodiment of at least Wash condition 3: 1.06×SSC, 0.1% SDS, 0% formamide at 60° C., in another embodiment of at least Wash condition 4: 1.06×SSC, 0.1% SDS, 0% formamide at 65° C., in another embodiment of at least Wash condition 5: 0.52×SSC, 0.1% SDS, 0% formamide at 65° C., in another embodiment of at least Wash condition 6: 0.25×SSC, 0.1% SDS, 0% formamide at 65° C., in another embodiment of at least Wash condition 7: 0.12×SSC, 0.1% SDS, 0% formamide at 65° C., in another embodiment of at least Wash condition 8: 0.07×SSC, 0.1% SDS, 0% formamide at 65° C.

A “low stringent wash” has stringency conditions comparable to the stringency conditions of at least Wash condition 1, but not more stringent than Wash condition 3, wherein the wash conditions are as described above.

A “high stringent wash” has stringency conditions comparable to the stringency conditions of at least Wash condition 4, in another embodiment of at least Wash condition 5, in another embodiment of at least Wash condition 6, in another embodiment of at least Wash condition 7, in another embodiment of at least Wash condition 8, wherein the wash conditions are as described above.

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EDNAFULL).

The following example is meant to illustrate two nucleotide sequences, but the same calculations apply to protein sequences:

Seq A: AAGATACTG length: 9 bases

Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, % identity is: for Seq A being the sequence of the invention (6/9)*100=66.7%; for Seq B being the sequence of the invention (6/8)*100=75%.

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 12 includes, by way of example, such nucleic acid sequences in cells which ordinarily contain SEQ ID NO:12 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

A “promoter” or “promoter sequence” or “regulatory nucleic acid” is a nucleotide sequence located upstream of a gene on the same strand as the gene that enables that gene's transcription. Promoter is followed by the transcription start site of the gene. Promoter is recognized by RNA polymerase (together with any required transcription factors), which initiates transcription. A functional fragment or functional variant of a promoter is a nucleotide sequence which is recognizable by RNA polymerase, and capable of initiating transcription.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Reduced expression: “reduce” or “lower” the expression of a nucleic acid molecule in a cell are used equivalently herein and mean that the level of expression of the nucleic acid molecule in a cell after applying a method of the present invention is lower than its expression in the cell before applying the method, or compared to a reference cell lacking a recombinant nucleic acid molecule of the invention. For example, the reference cell is comprising the same construct which is comprising the starting regulatory nucleic acid molecule and not the synthetic regulatory nucleic acid molecule of the invention. The term “reduced” or “lowered” as used herein are synonymous and means herein reduced, preferably significantly reduced expression of the nucleic acid molecule to be expressed. As used herein, an “reduction” of the level of an agent such as a protein, mRNA or RNA means that the level is reduced relative to a substantially identical cell grown under substantially identical conditions, lacking a recombinant nucleic acid molecule of the invention, for example comprising the starting regulatory nucleic acid molecule and not the synthetic regulatory nucleic acid molecule of the invention. As used herein, “reduction” of the level of an agent, such as for example a preRNA, mRNA, rRNA, tRNA, snoRNA, snRNA expressed by the target gene and/or of the protein product encoded by it, means that the level is reduced 10% or more, for example 20% or more, 30% or more, 40% or more, preferably 50% or more, for example 60% or more, 70% or more, 80% or more, 90% or more relative to a cell lacking a recombinant nucleic acid molecule of the invention, for example comprising the starting regulatory nucleic acid molecule and not the synthetic regulatory nucleic acid molecule of the invention. The reduction can be determined by methods with which the skilled worker is familiar. Thus, the reduction of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a cell. Depending on the type of the reduced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G250 (Bradford M M (1976) Analyt Biochem 72:248-254).

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target region” as used herein means the region close to, for example 10 bases, 20 bases, 30 bases, 40 bases, 50 bases, 60 bases, 70 bases, 80 bases, 90 bases, 100 bases, 125 bases, 150 bases, 200 bases or 500 bases or more away from the target site, or including the target site in which the sequence of the donor DNA molecule is introduced into the genome of a cell.

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention.

### FIGURES

FIG. 1 The plasmid map of the single CRISPR/Cas9 plasmid pCC009 is depicted. The plasmid pCC009 is a derivative of the plasmid pJOE8999.1 carrying the spacer for the amyB gene of Bacillus licheniformis and the DNA donor sequences HomA and HomB 5′ and 3′ of the amyB gene respectively. PmanP: promoter of the Bacillus subtilis manP gene, pUC ORI: high-copy origin of replication E. coli, Kanamycin resistance gene functional in both Bacillus and E. coli, rep pE194: fragment of plasmid pE194 conferring temperature-sensitive plasmid replication in Bacillus, PvanP: promoter driving expression of the spacer-sgRNA (crRNA repeat+rgRNA), TO terminator from lambda, t1 t2 terminators from the E. coli rrnB gene, HomA and HomB: sequences 5′ and 3′ of the amyB gene fused together for gene deletion, Cas9: Cas9 endonuclease from S. pyogenes.

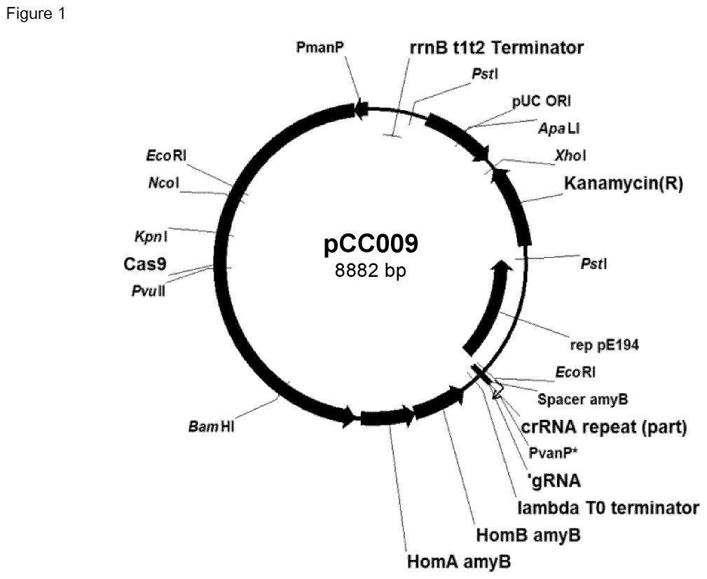

FIG. 2: The sequence alignment of selected regions of the mutated promoter sequences is shown—referenced against nt 15 to nt.128 of promoter sequences PV4 (SEQ ID 028) and PV8 (SEQ ID 029). Within the reference promoter sequences for the PV4 (SEQ ID 028) and PV8 (SEQ ID 029) promoters, the −35 and the −10 regions, the transcriptional start sites (TSS) and the Shine Dalgarno sequence (SD) are depicted in italic letters and shaded in grey. Nucleotide deletions, insertions and mutations are depicted in bold letters.

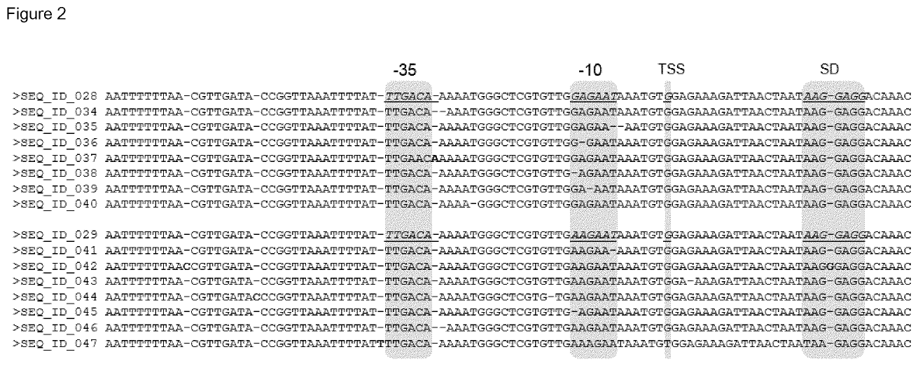

FIG. 3 Single colonies were analyzed by colony-PCR for deletion of the amyB gene of Bacillus licheniformis with oligonucleotides SEQ ID 009 and SEQ ID 010 lying outside the homology regions used for gene deletion. The gene deletion efficiency of the amylase amyB gene of Bacillus licheniformis as the percentage of clones with inactivated amylase gene relative to total of 20 clones analyzed for each gene deletion construct is plotted for each gene deletion construct as indicated. A. depicts the relative deletion efficiency of deletion plasmids derived from PV4 promoter variants. B. depicts the relative deletion efficiency of deletion plasmids derived from PV8 promoter variants.

FIG. 4 A. the gene deletion efficiency of the hag gene of Bacillus licheniformis as the percentage of clones with inactivated hag gene relative to total of 20 clones analyzed is plotted for two deletion constructs and promoter variants respectively as indicated. The average of three independent experiments with standard deviation is shown. The gene deletion of the hag gene was analyzed by colony PCR with oligonucleotides SEQ ID 087 and SEQ ID 088 lying outside the homology regions used for gene deletion. B. depicts the relative mutation efficiency of two deletion constructs and promoter variants respectively for introduction of point mutations within the degU gene of Bacillus licheniformis as the percentage of clones with mutated degU gene relative to total of 20 clones analyzed. The average of three independent experiments with standard deviation is shown. The gene mutation of the degU gene was analyzed by colony PCR with oligonucleotides SEQ ID 089 and SEQ ID 090 lying outside the homology region used for the introduction of the gene mutation, following restriction of the PCR fragment by Pstl to differentiate between native and mutated degU locus.

FIG. 5 A. the gene deletion efficiency of the amylase amyE gene of Bacillus subtilis as the percentage of clones with inactivated amyE gene relative to total of 20 clones analyzed is plotted for two deletion constructs and promoter variants respectively as indicated. The average of three independent experiments with standard deviation is shown. The gene deletion of the amyE gene was analyzed by colony PCR with oligonucleotides SEQ ID 091 and SEQ ID 092 lying outside the homology regions used for gene deletion. B. depicts the relative deletion efficiency of two deletion constructs and promoter variants respectively for deletion of the Subtilisin protease aprE gene of Bacillus subtilis as the percentage of clones with inactivated aprE gene relative to total of 20 clones analyzed. The average of three independent experiments with standard deviation is shown. The gene deletion of the aprE gene was analyzed by colony PCR with oligonucleotides SEQ ID 093 and SEQ ID 094 lying outside the homology regions used for gene deletion.

FIG. 6 A. the gene deletion efficiency of the vpr gene of Bacillus licheniformis as the percentage of clones with inactivated vpr gene relative to total of 20 clones analyzed is plotted for three deletion constructs and spacer variants respectively as indicated. The gene deletion of the vpr gene was analyzed by colony PCR with oligonucleotides SEQ ID 095 and SEQ ID 096 lying outside the homology regions used for gene deletion. B. depicts the relative deletion efficiency of three deletion constructs and spacer variants respectively for deletion of the epr gene of Bacillus licheniformis as the percentage of clones with inactivated epr gene relative to total of 20 clones analyzed. The gene deletion of the epr gene was analyzed by colony PCR with oligonucleotides SEQ ID 097 and SEQ ID 098 lying outside the homology regions used for gene deletion.

FIG. 7 The gene integration efficiency of the PaprE-GFPmut2 expression cassette replacing the amyB gene of Bacillus licheniformis as the percentage of clones with integrated PaprEGFPmut2 expression cassette relative to total of 20 clones analyzed is plotted for two different Bacillus licheniformis strains Bli #005 and P308 respectively as indicated. The average of two independent experiments with standard deviation is shown The integration was analyzed by colony PCR with oligonucleotides SEQ ID 009 and SEQ ID 010 lying outside the homology regions used for gene integration.

FIG. 8 The gene deletion efficiencies of the sporulation genes sigE, sigF and spollE of Bacillus pumilus as the percentage of clones with inactivated sporulation genes relative to total of 20 clones for each sporulation gene analyzed is plotted as indicated. The gene deletions of the sigE, sigF and spollE genes were analyzed by colony PCR with oligonucleotides SEQ ID 099 and SEQ ID 100, SEQ ID 101 and SEQ ID 102 and SEQ ID 103 and SEQ ID 104 respectively lying outside the homology regions used for gene deletion.

### EXAMPLES

Material and Methods

The following examples only serve to illustrate the invention. The numerous possible variations that are obvious to a person skilled in the art also fall within the scope of the invention. Unless otherwise stated the following experiments have been performed by applying standard equipment, methods, chemicals, and biochemicals as used in genetic engineering and fermentative production of chemical compounds by cultivation of microorganisms. See also Sambrook et al. (Sambrook, J. and Russell, D. W. Molecular cloning. A laboratory manual, 3rd ed, Cold Spring Harbor Laboratory Press, Cold Spring Harbor, N.Y. 2001) and Chmiel et al. (Bioprocesstechnik 1. Einfuhrung in die Bioverfahrenstechnik, Gustav Fischer Verlag, Stuttgart, 1991).

Electrocompetent Bacillus licheniformis cells and electroporation Transformation of DNA into Bacillus licheniformis strain DSM641 and ATCC53926 is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanpera J., 1989, FEMS Microbio. Lett., 61: 165-170) following plating on selective LB-agar plates.

In order to overcome the Bacillus licheniformis specific restriction modification system of Bacillus licheniformis strains DSM641 and ATCC53926, plasmid DNA is isolated from Ec #098 cells as described below. For transfer into Bacillus licheniformis restrictase knockout strains, plasmid DNA is isolated from E. coli INV110 cells (Life technologies).

Electrocompetent Bacillus pumilus Cells and Electroporation

Transformation of DNA into Bacillus pumilus DSM14395 is performed via electroporation. Preparation of electrocompetent Bacillus pumilus DSM14395 cells and transformation of DNA is performed as described for Bacillus licheniformis cells.

In order to overcome the Bacillus pumilus specific restriction modification system plasmid DNA is isolated from E. coli DH10B cells and plasmid DNA is in vitro methylated with whole cell extracts from Bacillus pumilus DSM14395 according to the method as described for Bacillus licheniformis in patent DE4005025.

Electrocompetent Bacillus subtilis Cells and Electroporation

Transformation of DNA into Bacillus subtilis ATCC6051a is performed via electroporation as described for Bacillus licheniformis and Bacillus pumilus respectively. Plasmid DNA isolated from E. coli DH10B cells can be readily used for transfer into Bacillus subtilis.

Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, N.Y. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37 C prior to cell lysis.

Annealing of Oligonucleotides to Form Oligonucleotide-Duplexes.

Oligonucleotides were adjusted to a concentration of 100 μM in water. 5 μl of the forward and 5 μl of the corresponding reverse oligonucleotide were added to 90 μl 30 mM Hepes-buffer (pH 7.8). The reaction mixture was heated to 95° C. for 5 min following annealing by ramping from 95° C. to 4° C. with decreasing the temperature by 0.1° C./sec (Cobb, R. E., Wang, Y., & Zhao, H. (2015). High-Efficiency Multiplex Genome Editing of Streptomyces Species Using an Engineered CRISPR/Cas System. ACS Synthetic Biology, 4(6), 723-728).

Molecular Biology Methods and Techniques

Standard methods in molecular biology not limited to cultivation of Bacillus and E. coli microorganisms, electroporation of DNA, isolation of genomic and plasmid DNA, PCR reactions, cloning technologies were performed as essentially described by Sambrook and Rusell. (Sambrook, J. and Russell, D. W. Molecular cloning. A laboratory manual, 3rd ed, Cold Spring Harbor Laboratory Press, Cold Spring Harbor, N.Y. 2001.)

Strains

E. coli Strain Ec #098

E. coli strain Ec #098 is an E. coli INV110 strain (Life technologies) carrying the DNAmethyltransferase encoding expression plasmid pMDS003 WO2019016051.

Generation of Bacillus licheniformis Gene k.o Strains

For gene deletion in Bacillus licheniformis strains DSM641 and ATCC53926 (U.S. Pat. No. 5,352,604) and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and used for subsequent transfer into Bacillus licheniformis strains. The isolated plasmid DNA carries the DNA methylation pattern of Bacillus licheniformis strains DSM641 and ATCC53926 respectively and is protected from degradation upon transfer into B. licheniformis.

B. licheniformis P304: Deleted Restriction Endonuclease

Electrocompetent Bacillus licheniformis DSM641 cells (U.S. Pat. No. 5,352,604) were prepared as described above and transformed with 1 μg of pDel006 restrictase gene deletion plasmid isolated from E. coli Ec #098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. driving integration of the deletion plasmid via Campbell recombination into the chromosome with one of the homology regions of pDel006 homologous to the sequences 5′ or 3′ of the aprE gene. Clones were picked and cultivated in LB-media without selection pressure at 45° C. for 6 hours, following plating on LB-agar plates with 5 μg/ml erythromycin at 30° C. Individual clones were picked and screened by colony-PCR analysis with oligonucleotides SEQ ID 014 and SEQ ID 015 for successful genomic deletion of the restrictase gene. Putative deletion positive individual clones were picked and taken through two consecutive overnight incubation in LB media without antibiotics at 45° C. to cure the plasmid and plated on LB-agar plates for overnight incubation at 37° C. Single clones were analyzed by colony PCR for successful genomic deletion of the restrictase gene. A single erythromycin-sensitive clone with the correct deleted restrictase gene was isolated and designated Bacillus licheniformis P304.

B. licheniformis P308: Deleted Poly-Gamma Glutamate Synthesis Genes

Electrocompetent Bacillus licheniformis P304 cells were prepared as described above and transformed with 1 μg of pDel007 pga gene deletion plasmid isolated from E. coli INV110 cells (Life technologies) following plating on LB-agar plates containing 5 μg/ml erythromycin at 30° C.

The gene deletion procedure was performed as described for the deletion of the restrictase gene.

The deletion of the pga genes was analyzed by PCR with oligonucleotides SEQ ID 017 and SEQ ID 018 The resulting Bacillus licheniformis strain with deleted pga synthesis genes was named Bacillus licheniformis P308.

B. licheniformis Bli #002: Deleted aprE Gene

Electrocompetent Bacillus licheniformis ATCC53926 cells were prepared as described above and transformed with 1 μg of pDel003 aprE gene deletion plasmid isolated from E. coli Ec #098 following plating on LB-agar plates containing 5 μg/ml erythromycin at 30° C. The gene deletion procedure was performed as described for the deletion of the restrictase gene. The deletion of the aprE gene was analyzed by PCR with oligonucleotides SEQ ID 020 and SEQ ID 021 The resulting Bacillus licheniformis strain with deleted aprE gene was named Bli #002.

B. licheniformis Bli #005: Deleted Poly-Gamma Glutamate Synthesis Genes

The poly-gamma-glutamate synthesis genes were deleted in Bacillus licheniformis Bli #002 as described for the deletion of the pga genes in Bacillus licheniformis P304 with the difference that the pDel007 plasmid was isolated from E. coli Ec #098 cells. The resulting strain was named Bli #005.

Plasmids

pEC194RS—Bacillus Temperature Sensitive Deletion Plasmid.

The plasmid pE194 is PCR-amplified with oligonucleotides SEQ ID 001 and SEQ ID 002 with flanking Pvull sites, digested with restriction endonuclease Pvull and ligated into vector pCE1 digested with restriction enzyme Smal. pCE1 is a pUC18 derivative, where the Bsal site within the ampicillin resistance gene has been removed by a silent mutation. The ligation mixture was transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200) (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) with oligonucleotides SEQ ID 003 and SEQ ID 004, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprE gene of Bacillus licheniformis was constructed with plasmid pEC194RS and the gene synthesis construct SEQ ID 019 comprising the genomic regions 5′ and 3′ of the aprE gene flanked by Bsal sites compatible to pEC194RS. The type-II-assembly with restriction endonuclease Bsal was performed as described (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) and the reaction mixture subsequently transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting aprE deletion plasmid is named pDel003.

pDel006—Restrictase Gene Deletion Plasmid

The gene deletion plasmid for the restrictase gene (SEQ ID 012) of the restriction modification system of Bacillus licheniformis DSM641 (SEQ ID 011) was constructed with plasmid pEC194RS and the gene synthesis construct SEQ ID 013 comprising the genomic regions 5′ and 3′ of the restrictase gene flanked by Bsal sites compatible to pEC194RS. The type-II-assembly with restriction endonuclease Bsal was performed as described above and the reaction mixture subsequently transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting restrictase deletion plasmid is named pDel006.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel006, however the gene synthesis construct SEQ ID 016 comprising the genomic regions 5′ and 3′ flanking the ywsC, ywtA (pgsC), ywtB (pgsA), ywtC (pgsE) genes flanked by Bsal sites compatible to pEC194RS was used. The resulting pga deletion plasmid is named pDel007.

Plasmid p689-T2A-lac

The plasmid p689-T2A-lac comprises the lacZ-alpha gene flanked by Bpil restriction sites, again flanked 5′ by the T1 terminator of the E. coli rrnB gene and 3′ by the TO lambda terminator and was ordered as gene synthesis construct (SEQ ID 073).

Plasmid p890 PaprE-GFPmut2

The promoter of the aprE gene from Bacillus licheniformis of plasmid pCB56C (U.S. Pat. No. 5,352,604) was PCR-amplified with oligonucleotides SEQ ID 074 and SEQ ID 075. The GFPmut2 gene variant (accession number AF302837) with flanking Bpil restriction sites (SEQ ID 076) was ordered as gene synthesis fragment (Geneart Regensburg). The gene expression construct comprising the PaprE promoter from Bacillus licheniformis fused to the GFPmut2 variant was cloned into plasmid p689-T2A-lac by type-II-assembly with restriction endonuclease Bpil as described (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) and the reaction mixture subsequently transformed into electrocompetent E. coli DH10B cells. Transformants were spread and incubated overnight at 37° C. on LB-agar plates containing 100 μg/ml ampicillin. Plasmid DNA was isolated from individual clones and analyzed for correctness by restriction digest and sequencing. The resulting plasmid is named p890 PaprE-GFPmut2.

Plasmid pJOE8999.1:

Altenbuchner J. 2016. Editing of the Bacillus subtilis genome by the CRISPR-Cas9 system. Appl Environ Microbiol 82:5421-5.

Plasmid pJOE-T2A

To allow for type-II-assembly (T2A) based one-step-cloning of the sgRNA and the homology regions for DSB repair the CRISPR/Cas9 plasmid pJOE8889.1 was modified as follows. The type-II-assembly mRFP cassette from plasmid pBSd141R (accession number: KY995200) (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) was modified such to remove multiple restriction sites and the Bpil restriction sites and ordered as gene synthesis fragment with flanking Sfil restriction sites (SEQ ID 005). The plasmid is named p #732. Plasmid p #732 and plasmid pJOE8999.1 were digested with Sfil (New England Biolabs, NEB) and the mRFP cassette of p #732 ligated into Sfil-digested pJOE8999.1 following transformation into competent E. coli DH10B cells. Positive clones were screened on IPTG/X-Gal and kanamycin (20 μg/ml) containing LB agar plates for purple colonies (blue-white screening and mRFP1 expression). The resulting sequence-verified plasmid was named pJOE-T2A.

Plasmid pBW732

The 5′ homology region (also referred to as HomA) and the 3′ homology region (also referred to as HomB) adjacent to the amylase amyB gene of Bacillus licheniformis DSM641 was ordered as synthetic gene synthesis fragment with flanking Xmal restriction sites (SEQ ID 006). The plasmid pJOE8999.1 and the synthetic amyB-HomAB fragment are cleaved with restriction endonuclease Xmal following ligation with T4-DNA ligase (NEB) and transformation into electrocompetent E. coli DH10B cells. The correct plasmid was recovered and named pBW732.

Plasmid pBW742

The 20 bp target sequence of the amyB gene for the sgRNA was designed using Geneious 11.1.5 (https://www.geneious.com). The resulting oligonucleotides SEQ ID 007 and Seq ID 008 with 5′ phosphorylation were annealed to form an oligonucleotide duplex. The CRISPR/Cas9 based gene deletion plasmid for the amyB gene of Bacillus licheniformis was constructed by type-II-assembly with restriction endonuclease Bsal as described (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) with the following components: pBW732 and the oligonucleotide duplex (SEQ ID 007, SEQ ID 008). The reaction mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting amyB deletion plasmid is named pBW742.

T2A CRISPR Destination Vectors pCCO27 and pCCO28

Plasmid pCC014 and pCCO25 were modified such that region covering the spacer-sgRNA and amyB gene flanking homologous regions were replaced by the T2A cassette from plasmid pJOE-T2A. The backbones of pCC014 and pCCO25 were PCR amplified with oligonucleotides SEQ ID 050 and SEQ ID 051 and the T2A assembly cassette was PCR-amplified from pJOE-T2A with oligonucleotides SEQ ID 048 and SEQ ID 049 following PCR purification using the High Pure PCR purification Kit, digestion with Dpnl and gel purification. The corresponding backbone PCR fragments and the T2A cassette PCR fragment were annealed in a 10 μl Gibson reaction following transformation into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting pCC014 and pCCO25 derived T2A plasmid derivatives are designated pCCO27 and pCCO28 respectively.

pCCO29—Hag Gene Deletion Plasmid

The 20 bp target sequence of the hag gene for the sgRNA was designed using Geneious 11.1.5 as described before. The resulting oligonucleotides SEQ ID 056 and Seq ID 057 with 5′ phosphorylation were annealed to form an oligonucleotide duplex as described above. The genomic regions 5′ and 3′ of the hag gene were PCR-amplified on genomic DNA from Bacillus licheniformis DSM641 with oligonucleotides SEQ ID 054 and Seq ID 053 and SEQ ID 052 and Seq ID 55 following fusion by overlap extension PCR with flanking oligonucleotides SEQ ID 053 and SEQ ID 054. The resulting PCR product was column purified (Qiagen PCR purification Kit). The CRISPR/Cas9 based gene deletion plasmid for the hag gene of Bacillus licheniformis was constructed by type-II-assembly with restriction endonuclease Bsal as described before with the following components: plasmid pCCO27 (PV4-5 promoter variant), the fused homology regions of the hag gene with flanking Bsal restriction sites and the oligonucleotide duplex (SEQ ID 056, SEQ ID 057). The reaction mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting hag gene deletion plasmid is named pCCO29.

pCC030—Hag Gene Deletion Plasmid

The hag gene deletion construct was constructed as for pCCO29 however the plasmid pCCO28 (PV8-7 promoter variant) was used.

pCC031—degU32 Gene Editing Plasmid

The construction of the degU32 genome editing construct to introduce the degU H12L mutation was performed as for pCCO29 with the following modifications.

The degU32 homology regions introducing the mutations for the degU H12L mutation as well as the introduction of a silent point mutation to remove the PAM site were ordered as gene synthesis construct (Geneart, Regensburg) with flanking Bsal sites (SEQ ID 058). The 20 bp target sequence of the degU gene for the sgRNA was designed and the resulting oligonucleotides SEQ ID 059 and Seq ID 060 with 5′ phosphorylation were annealed to form an oligonucleotide duplex as described above.

pCC032—degU32 Gene Editing Plasmid

The degU32 genome editing construct was made as described for pCC031 however the plasmid pCCO28 (PV8-7 promoter variant) was used.

pCC033—amyE Gene Deletion Plasmid

The fragment comprising the amyE spacer-sgRNA and homology regions of the 5′ and 3′ regions of the amyE gene from Bacillus subtilis was PCR-amplified from plasmid pCC004 (WO17186550) with oligonucleotides SEQ ID 061 and SEQ ID 062 with flanking Bsal restriction sites. The CRISPR/Cas9 based gene deletion plasmid for the amylase amyE gene was subsequently constructed by type-II-assembly with restriction endonuclease Bsal as described above with plasmid pCCO27 (PV4-5 promoter variant) and the PCR-amplified fragment. The reaction mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting amyE gene deletion plasmid is named pCC033.

pCC034—amyE Gene Deletion Plasmid

The amyE gene deletion construct was constructed as for pCC033, however the plasmid pCCO28 (PV8-7 promoter variant) was used.

pCC035—aprE Gene Deletion Plasmid

The fragment comprising the aprE spacer (SEQ ID 064)-sgRNA and homology regions of the 5′ and 3′ regions of the aprE gene of Bacillus subtilis was ordered as synthetic gene fragment (SEQ ID 063) with flanking Bsal restriction sites. The CRISPR/Cas9 based gene deletion plasmid for the protease aprE gene was subsequently constructed by type-II-assembly with restriction endonuclease Bsal as described above with plasmid pCCO27 (PV4-5 promoter variant) and gene synthesis construct. The reaction mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting aprE gene deletion plasmid is named pCC035.

pCC036—aprE Gene Deletion Plasmid

The aprE gene deletion construct was constructed as for pCC035, however the plasmid pCCO28 (PV8-7 promoter variant) was used.

pCC037—pCC039—vpr Gene Deletion Plasmids

The CRISPR/Cas9 gene deletion constructs pCC037, pCC038 and pCC039 of the protease vpr gene of Bacillus licheniformis were constructed as described for pCC035, however with synthetic gene fragments comprising the vpr spacer-sgRNA and homology regions of the 5′ and 3′ regions of the vpr gene (SEQ ID 065). The resulting plasmids pCC037, pCC038 and pCC039 differ in the vpr spacer sequences (SEQ ID 066, SEQ ID 067, SEQ ID 068) within SEQ ID 065.

pCC040—pCC042—epr Gene Deletion Plasmids

The CRISPR/Cas9 gene deletion constructs pCC040, pCC041 and pCC042 of the protease epr gene of Bacillus licheniformis were constructed as described for pCC035, however with synthetic gene fragments comprising the epr spacer-sgRNA and homology regions of the 5′ and 3′ regions of the epr gene (SEQ ID 069). The resulting plasmids pCC040, pCC041 and pCC042 differ in the epr spacer sequences (SEQ ID 070, SEQ ID 071, SEQ ID 072) within SEQ ID 069.

pCC043—GFP Gene Integration Plasmid

The 20 bp target sequence of the amyB gene for the sgRNA were ordered as oligonucleotides SEQ ID 007 and Seq ID 008 with 5′ phosphorylation following annealing to form an oligonucleotide duplex. The 5′ and 3′ regions of the amyB gene of Bacillus licheniformis were PCR-amplified with oligonucleotides SEQ ID 077 and SEQ ID 078 and SEQ ID 079 and SEQ ID 080 respectively.

The CRISPR/Cas9 based gene integration plasmid replacing the amyB gene of Bacillus licheniformis was constructed by type-II-assembly with restriction endonuclease Bsal as described as described above with the following components: pCCO27, the oligonucleotide duplex (SEQ ID 007, SEQ ID 008), the PCR-fragment of the 5′ homology region of the amyB gene, p890-PaprE-GFPmut2 and the PCR-fragment of the 3′ homology regions of the amyB gene. The reaction mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest and sequencing. The resulting CRISPR/Cas9 based gene integration plasmid is named pCC043.

pCC044—sigE Gene Deletion Plasmid Bacillus pumilus

The CRISPR/Cas9 gene deletion construct pCC044 of the sigE gene of Bacillus pumilus DSM14395 was constructed as described for pCC035, however with a synthetic gene fragment (SEQ ID 082) comprising the sigE spacer (SEQ ID 081)-sgRNA and homology regions of the 5′ and 3′ regions of the sigE gene.

pCC045—sigF Gene Deletion Plasmid Bacillus pumilus

The CRISPR/Cas9 gene deletion construct pCC045 of the sigF gene of Bacillus pumilus DSM14395 was constructed as described for pCC035, however with a synthetic gene fragment (SEQ ID 084) comprising the sigF spacer (SEQ ID 083)-sgRNA and homology regions of the 5′ and 3′ regions of the sigF gene.

pCC046—spollE Gene Deletion Plasmid Bacillus pumilus

The CRISPR/Cas9 gene deletion construct pCC046 of the spollE gene of Bacillus pumilus DSM14395 was constructed as described for pCC035, however with a synthetic gene fragment (SEQ ID 086) comprising the spollE spacer (SEQ ID 085)-sgRNA and homology regions of the 5′ and 3′ regions of the spollE gene.

### Example 1: Construction of CRISPR/Cas9 Genome Editing Plasmids with Constitutive Promoter

In order to introduce a constitutive promoter driving the expression of the Cas9 enzyme in plasmid pBW742 a two-step procedure was applied.

Frist, the t1t2t0 terminator (derived from pMUTIN) was introduced 5′ of the promoter PmanP of pBW742 to prevent potential read-through from the kanamycin selection marker.

The terminator sequence t1t2t0 was integrated into pBW742 upstream of the mannose promoter by Gibson assembly (NEBuilder® HiFi DNA Assembly Cloning Kit, New England Biolabs). To this purpose, the terminator fragment (0.44 kb) was amplified by PCR with oligonucleotides SEQ ID 024 and SEQ ID 025 using pMutin2 (accession number AF072806) as the template. The corresponding vector backbone of pBW742 was amplified with oligonucleotides SEQ ID 022 and SEQ ID 023. The pBW742 amplicon was purified using the PCR product purification kit (Roche). After subsequent digestion of the pBW742 PCR product with Dpnl (New England Biolabs), both PCR fragments were gel purified using the Qiaquick Gel Extraction Kit (Qiagen, Hilden, Germany) and annealed in a 1:2 ratio for 1 h at 50° C. E. coli strain DH10B was transformed with the assembly reaction following plating on LB-agar plates containing 20 μg/ml kanamycin. Plasmid DNA was isolated from individual clones and analyzed for correctness by restriction digest and sequencing.

A deviation from the published reference sequence of pMutin2 was found. The SEQ ID 026 covers the part of the pMutin2 sequence, SEQ ID 027 covers the sequence deviation found within the corresponding region of pMutin2 found in the resulting plasmid pCC009.

Secondly, the mannose-inducible promoter PmanP was exchanged by two promoter variants of the constitutive promoter Pveg from Bacillus subtilis—namely PV4 and PV8-derived from Guiziou et al (Guiziou, S., V. Sauveplane, H. J. Chang, C. Clerte, N. Declerck, M. Jules, and J. Bonnet. 2016. A part toolbox to tune genetic expression in Bacillus subtilis. Nucleic Acids Res. 44: 7495-7508). These promoter variants which comprise the Pveg promoter, a standardized TSS (transcriptional start site) region and the standardized ribosome binding site region R0, derived from the adapted Pveg promoter library that was screened on single copy level in Bacillus subtilis with regards to their altered expression levels. The PV4 and PV8 promoter sequences are listed as SEQ ID 028 and SEQ ID 029 respectively.

The integration of both promoter variants was carried out by Gibson assembly. Amplification of the PV4 and PV8 fragments was done stepwise. For both promoter fragments, using pCC009 as the template, oligonucleotides SEQ ID 024 and SEQ ID 030 were used for the first PCR (Phusion high fidelity DNA polymerase—NEB) and the resulting products served as the template for a second PCR with the oligonucleotides SEQ ID 024 and SEQ ID 031 for PV4 and SEQ ID 024 and SEQ ID 033 for PV8.

The vector backbone of pCC009 was PCR amplified using oligonucleotides SEQ ID 022 and SEQ ID 032. After purification of the vector amplicon with the PCR purification kit (Roche), PCR product digestion with Dpnl was carried out to remove remaining circular plasmid DNA from the PCR reaction. Subsequently, the digested vector and both promoter fragments were purified using the Qiaquick Gel Extraction Kit (Qiagen, Hilden, Germany). The vector amplicon of pCC009 was then annealed with the promoter fragments of PV4 and PV8, respectively, thereby replacing the mannose promoter PmanP with the PV4 and PV8 variants of the Pveg promoter.

The annealing reactions were subsequently transformed into E. coli DH10B cells (Life technologies). Transformants were spread and incubated overnight at 37 C on LB-agar plates containing 20 μg/ml kanamycin. Plasmid DNA was isolated from 9 individual clones of PV4 promoter and 8 individual clones from promoter variant PV8 and analyzed for correctness by sequencing.

Table 1 Summarizes the Sequencing Results of the Various Promoter Variants:

Analysis of clones from PV4-cloning reactions reveals that only sequences with point mutations, nucleotide insertions or deletions within the PV4 region could be recovered.

Analysis of clones from PV8-cloning reactions reveals that that only sequences with point mutations, nucleotide insertions or deletions within the PV8 region could be recovered. The resulting plasmids are summarized in Table 1.

TABLE 1

| Plasmid | Promoter variant | SEQ ID |
|---|---|---|
| pCC010 | Pv4-1 | 034 |
| pCC011 | Pv4-2 | 035 |
| pCC012 | Pv4-3 | 036 |
| pCC013 | Pv4-4 | 036 |
| pCC014 | Pv4-5 | 037 |
| pCC015 | Pv4-6 | 038 |
| pCC016 | Pv4-7 | 039 |
| pCC017 | Pv4-8 | 040 |
| pCC018 | Pv4-9 | 039 |
| pCC019 | Pv8-1 | 041 |
| pCC020 | Pv8-2 | 042 |
| pCC021 | Pv8-3 | 043 |
| pCC022 | Pv8-4 | 044 |
| pCC023 | Pv8-5 | 045 |
| pCC024 | Pv8-6 | 041 |
| pCC025 | Pv8-7 | 046 |
| pCC026 | Pv8-8 | 047 |

Gene Deletion Efficiency of CRISPR/Cas9 Based Deletion Plasmids

Electrocompetent Bacillus licheniformis P308 cells were prepared as described above and transformed with 1 μg of amyB deletion plasmids pCC010-012, pCC014-017, pCC019-026 (with different promoter variants as depicted in Table 1) isolated from E. coli INV110 cells (Life technologies) following plating on LB-agar plates containing 20 μg/ml kanamycin and incubation overnight at 37° C.

The next day 20 clones of each transformation reaction were subjected to colony-PCR to analyze for successful CRISPR/Cas9 based deletion of the amyB gene and with oligonucleotides SEQ ID 009 and SEQ ID 010, and further transferred onto fresh LB-agar plates without antibiotics following incubation at 48° C. overnight for plasmid curing.

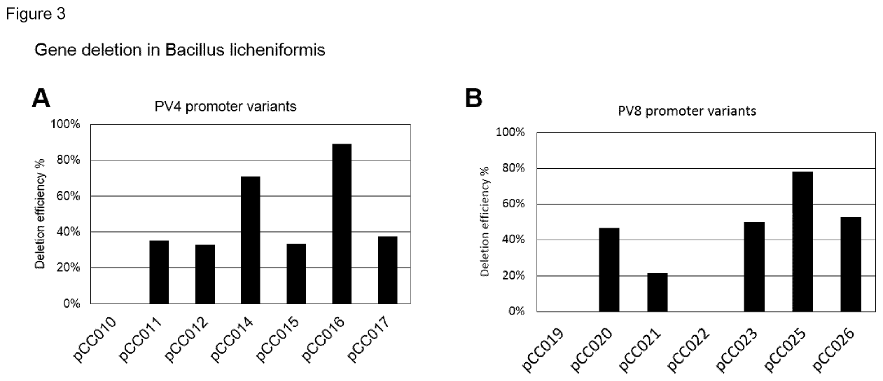

The efficiency of amyB gene deletion for each CRISPR/Cas9 based deletion plasmid was calculated as the ratio in percentage of successful gene deletion based on the appearance of the expected smaller specific PCR-amplicon compared to the larger specific PCR-amplicon of the wild-type amyB gene locus relative to the total number of clones analyzed. As depicted in FIG. 3 CRISPR/Cas9 based amyB gene deletion plasmids pCC010, pCC019 and pCCO22 are not functional in Bacillus licheniformis as all cells analyzed carried the wild-type amyB locus.

The other promoter variants are functional in Bacillus licheniformis driving the expression of Cas9. In particular, gene deletion plasmids pCC014, pCC016, pCCO25 with promoter variants PV4-5, PV4-7 and PV8-7 respectively show highest gene deletion efficiency with greater 60%.

A single correct clone was steaked onto fresh LB-agar plates without antibiotics following second incubation at 48° C. overnight for plasmid curing. Final clones were again analyzed for successful amyB gene deletion by colony PCR and plasmid loss analyzed by plating on LB-agar plates containing 20 μg/ml kanamycin. The resulting Bacillus licheniformis strain with cured deletion plasmid (sensitive to kanamycin) and deleted amyB gene was named Bacillus licheniformis P310.

### Example 2: Gene Deletion and Gene Mutation with Promoters PV4-5 and PV8-7 in Bacillus licheniformis

Electrocompetent Bacillus licheniformis P308 cells were prepared as described above and transformed with 1 μg of each of the hag deletion plasmids pCCO29 and pCC030 with promoters PV4-5 (SEQ ID 037) PV8-7 (SEQ ID 046) respectively isolated from E. coli INV110 cells (Life technologies) following plating on LB-agar plates containing 20 μg/ml kanamycin and incubation overnight at 37° C.

The next day 20 clones of each transformation reaction were subjected to colony-PCR to analyze for successful CRISPR/Cas9-based deletion of the hag gene and with oligonucleotides SEQ ID 087 and SEQ ID 088, and further transferred onto fresh LB-agar plates without antibiotics following incubation at 48° C. overnight for plasmid curing.

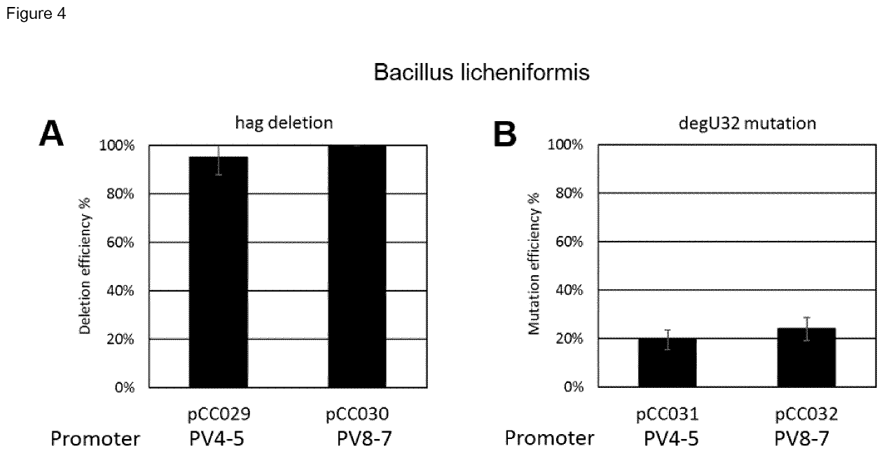

The efficiency of hag gene deletion for each CRISPR/Cas9-based deletion plasmid was calculated as the ratio in percentage of successful gene deletion based on the appearance of the expected smaller specific PCR-amplicon compared to the larger specific PCR-amplicon of the wild-type hag gene locus relative to the total number of clones analyzed. The experiment for each hag gene deletion plasmid was performed three times. As depicted in FIG. 4A the CRISPR/Cas9-based hag gene deletion efficiencies of plasmids pCCO29 and pCC030 are 95% and 100% respectively.

To analyze the efficiency for introduction of point mutations, Bacillus licheniformis P308 cells were transformed with two degU mutation plasmids pCC031 and pCC032 as described for deletion of the hag gene, again differing in the promoters PV4-5 (SEQ ID 037) and PV8-7 (SEQ ID 046) driving the constitutive expression of Cas9. The transformed Bacillus licheniformis cells were plated on LB-agar plates containing 20 μg/ml kanamycin following incubation overnight at 30° C. The mutation efficiency of introduction of the H12L degU mutation was calculated as the ratio in percentage of successful mutated degU gene based on the appearance of a degU-specific PCR-amplicon with oligonucleotides SEQ ID 089 and SEQ ID 090 that can be cleaved with the restriction endonuclease Pstl compared to the native degU-specific PCR-amplicon of the wild-type degU gene locus relative to the total number of 20 clones analyzed. The experiment for each degU mutation plasmid was performed three times. As depicted in FIG. 4B the CRISPR/Cas9-based mutation efficiencies of plasmids pCC031 and pCC032 are 19% and 24% respectively.

### Example 3: Gene Deletion with Promoters PV4-5 and PV8-7 in Bacillus subtilis

Electrocompetent Bacillus subtilis ATCC6051a cells were prepared as described above and transformed with 1 μg of each of the amyE deletion plasmids pCC033 and pCC034 with promoters PV4-5 (SEQ ID 037) and PV8-7 (SEQ ID 046) respectively isolated from E. coli DH10B cells following plating on LB-agar plates containing 20 μg/ml kanamycin and incubation overnight at 37° C.

The next day 20 clones of each transformation reaction were subjected to colony-PCR to analyze for successful CRISPR/Cas9-based deletion of the amyE gene with oligonucleotides SEQ ID 091 and SEQ ID 092, and further transferred onto fresh LB-agar plates without antibiotics following incubation at 48° C. overnight for plasmid curing.

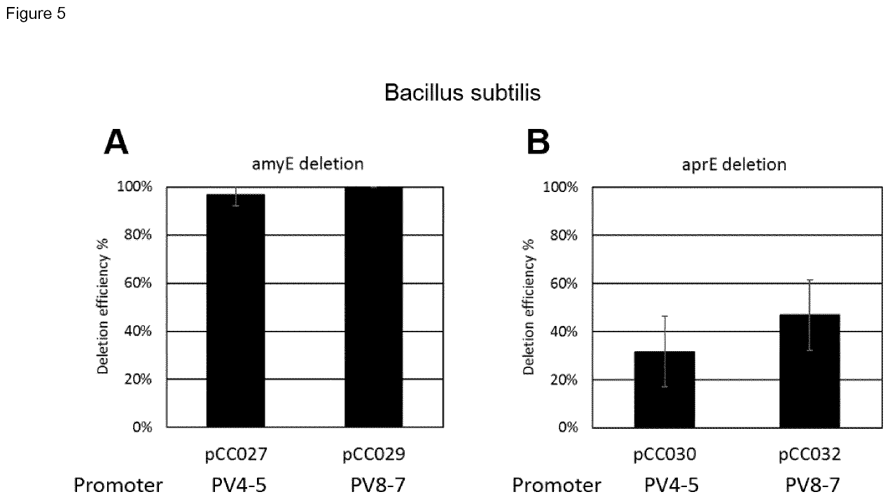

The efficiency of amyE gene deletion for each CRISPR/Cas9-based deletion plasmid was calculated as the ratio in percentage of successful gene deletion based on the appearance of the expected smaller specific PCR-amplicon compared to the larger specific PCR-amplicon of the wild-type amyE gene locus relative to the total number of clones analyzed. The experiment for each amyE gene deletion plasmid was performed three times. As depicted in FIG. 5A the CRISPR/Cas9-based amyE gene deletion efficiencies of plasmids pCC033 and pCC034 within Bacillus subtilis are 97% and 100% respectively.

The gene deletion efficiency of plasmids pCC035 and pCC036 in dependency of promotors PV4-5 (SEQ ID 037) and PV8-7 (SEQ ID 046) for deletion of the aprE gene of Bacillus subtilis was analyzed similar to the procedure described for the deletion of the amyE gene, however cells were incubated on LB-agar plates containing 20 μg/ml kanamycin after transformation at 30° C. overnight. The gene deletion was again analyzed by colony-PCR with oligonucleotides SED ID 093 and SEQ ID 094 and the gene deletion efficiency calculated as described above for three independent transformation reactions. As depicted in FIG. 5B the CRISPR/Cas9-based aprE gene deletion efficiencies of plasmids pCC035 and pCC036 within Bacillus subtilis are 32% and 47% respectively.

### Example 4: Gene Deletion with Promoters PV4-5 and PV8-7 and Different Spacers in Bacillus licheniformis

Electrocompetent Bacillus licheniformis Bli #005 cells were prepared as described above and transformed with 1 μg of each of the vpr deletion plasmids pCC037, pCC038 and pCC039 with promoter PV4-5 (SEQ ID 037) and different vpr-specific spacer sequences (SEQ ID 066-068) respectively isolated from E. coli Ec #098 cells following plating on LB-agar plates containing 20 μg/ml kanamycin and incubation overnight at 37° C.

The next day 20 clones of each transformation reaction were subjected to colony-PCR to analyze for successful CRISPR/Cas9-based deletion of the vpr gene with oligonucleotides SEQ ID 095 and SEQ ID 096, and further transferred onto fresh LB-agar plates without antibiotics following incubation at 48° C. overnight for plasmid curing.

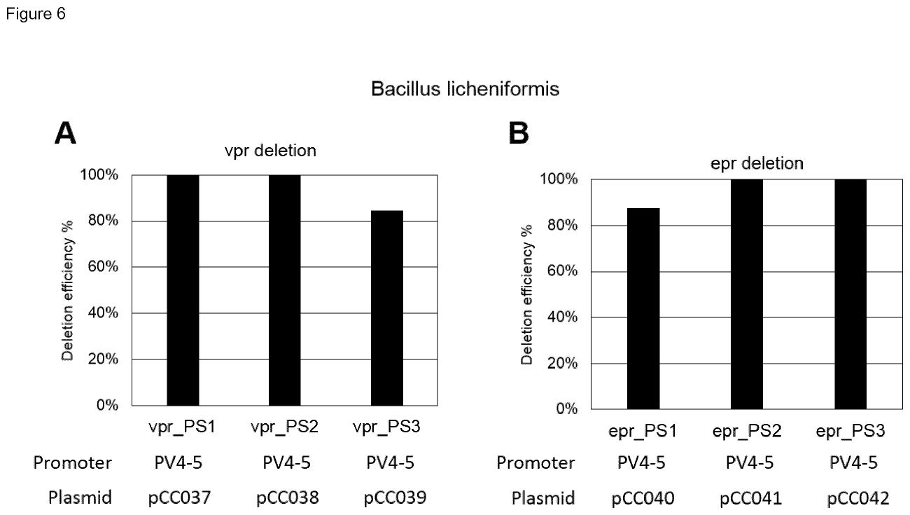

The efficiency of vpr gene deletion for each CRISPR/Cas9-based deletion plasmid was calculated as the ratio in percentage of successful gene deletion based on the appearance of the expected smaller specific PCR-amplicon compared to the larger specific PCR-amplicon of the wild-type vpr gene locus relative to the total number of clones analyzed. As depicted in FIG. 6A the CRISPR/Cas9-based vpr gene deletion efficiency of plasmids pCC037, pCC038 and pCC039 is 100%, 100% and 84% respectively.

The gene deletion efficiency of plasmids pCC040. pCC041 and pCC042 with promoter PV4-5 (SEQ ID 037) and different epr-specific spacer sequences (SEQ ID 070-072) for deletion of the epr gene of Bacillus licheniformis was done as described for the vpr gene, however, oligonucleotides SEQ ID 097 and SEQ ID098 were used for colony-PCR-based analysis of the gene deletion. As depicted in FIG. 6B the CRISPR/Cas9-based epr gene deletion efficiency of plasmids pCC040, pCC041 and pCC042 is 87.5%, 100% and 100% respectively.

### Example 5: Gene Integration with Promoters PV4-5 and PV8-7 in Bacillus licheniformis

Electrocompetent Bacillus licheniformis Bli #005 cells were prepared as described above and transformed with 1 μg of the gene integration plasmid pCC043 with promoter PV4-5 (SEQ ID 037) isolated from E. coli Ec #098 cells following plating on LB-agar plates containing 20 μg/ml kanamycin and incubation overnight at 37° C.

The next day 20 clones of the transformation reaction were subjected to colony-PCR with oligonucleotides SEQ ID 009 and SEQ ID 010 to analyze for successful CRISPR/Cas9-based integration of the PaprE-GFPmut2 expression cassette to replace the amyB gene of Bacillus licheniformis, and further transferred onto fresh LB-agar plates without antibiotics following incubation at 48° C. overnight for plasmid curing.

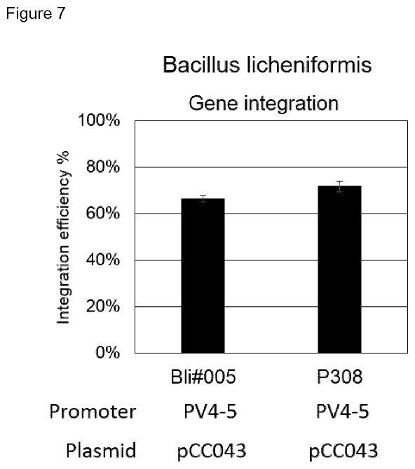

The efficiency of gene integration for the pCC043 CRISPR/Cas9-based gene integration plasmid was calculated as the ratio in percentage of successful gene integration based on the appearance of the expected specific PCR-amplicon compared to the larger specific PCR-amplicon of the wild-type amyB gene locus relative to the total number of clones analyzed. The experiment was performed twice. As depicted in FIG. 7 the CRISPR/Cas9-based gene integration efficiency of plasmid pCC043 into Bli #005 is 67%.

The efficiency of the gene integration of the PaprE-GFPmut2 expression cassette with plasmid pCC043 was similarly determined for the Bacillus licheniformis P308 strain showing in two independent transformation reactions an average gene integration efficiency of 72% as depicted in FIG. 7.

### Example 6: Gene Deletion with Promoters PV4-5 in Bacillus pumilus

Electrocompetent Bacillus pumilus DSM14395 cells were prepared as described above and transformed with 1 μg each of the sporulation gene deletion plasmids pCC044 (sigE), pCC045 (sigF) and pCC046 (spollE) with promoter PV4-5 (SEQ ID 037) driving the expression of the Cas9 endonuclease. The plasmid DNA was isolated from E. coli DH10B cells and in vitro methylated as described above prior to transformation. Transformed Bacillus pumilus cells were plated on LB-agar plates containing 20 μg/ml kanamycin and incubated overnight at 37° C.

The next day 20 clones of each of the transformation reactions were subjected to colony-PCR with oligonucleotides SEQ ID 099 and SEQ ID 100 for analysis of the sigE deletion, with oligonucleotides SEQ ID 101 and SEQ ID 102 for analysis of the sigF deletion and with oligonucleotides SEQ ID 103 and SEQ ID 104 for analysis of the spollE deletion. Individual colonies were further transferred onto fresh LB-agar plates without antibiotics following incubation at 48° C. overnight for plasmid curing.

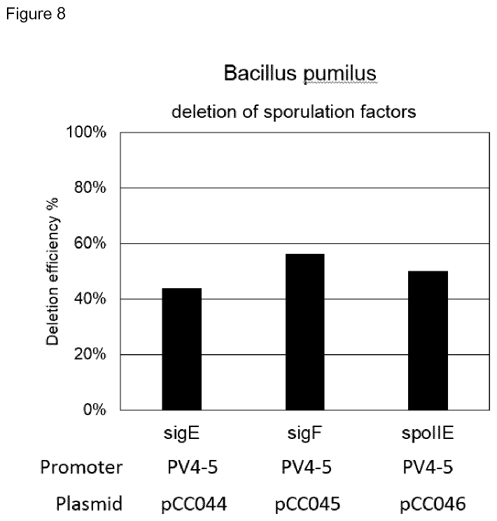

The efficiencies of the gene deletion of plasmids pCC044, pCC045 and pCC046 in Bacillus pumilus were calculated as the ratio in percentage of successful gene deletion based on the appearance of the expected smaller specific PCR-amplicon compared to the larger specific PCR-amplicon of the wild-type gene locus relative to the total number of clones analyzed. As depicted in FIG. 8 the CRISPR/Cas9-based gene deletion efficiencies of plasmids pCC044, pCC045 and pCC046 within Bacillus pumilus are 43%, 56% and 50% respectively.

1. A method for the production of one or more synthetic regulatory nucleic acid molecule conferring reduced constitutive expression compared to a respective starting regulatory nucleic acid molecule in a bacterial cell comprising the steps of
a. identifying at least one starting regulatory nucleic acid molecule conferring constitutive expression in a bacterial cell,
b. operably linking said starting regulatory nucleic acid molecule to a coding region encoding a protein heterologous to said starting regulatory nucleic acid molecule,
c. introducing the construct comprising said starting regulatory nucleic acid molecule operably linked to a coding region into a vector comprising an origin of replication conferring high copy numbers of said vector within a bacterial cell wherein said construct confers high expression of said coding region wherein high expression of said coding region in a bacterial cell burdens said bacterial cell leading to reduced or abolished growth,
d. transforming said vector into bacterial cells,
e. growing said transformed bacterial cells to recover single clones,
f. isolating single clones exhibiting growth rates comparable to corresponding bacterial strains not comprising said construct,
g. isolating from said clones said construct,
h. testing the synthetic regulatory nucleic acid molecule comprised in said construct for functional expression of a gene operably linked to said synthetic regulatory nucleic acid molecule, and optionally
i. sequencing the respective regulatory nucleic acid molecule comprised in said construct, thereby identifying a synthetic regulatory nucleic acid molecule conferring reduced constitutive expression in a bacterial cell, wherein the synthetic regulatory nucleic acid molecule is active in cells of the genus bacilli and the genus Escherichia.

2. The method of claim 1, wherein the synthetic regulatory nucleic acid molecule confers reduced expression in a bacterial cell distinct from the cell in which the recombinant nucleic acid is produced.

3-7. (canceled)

8. The method of claim 2, wherein the synthetic regulatory nucleic acid molecule is active in cells of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells.

9. The method of claim 8, wherein the synthetic regulatory nucleic acid molecule is active in cells of at least three different bacilli species.

10. The method of claim 9, wherein the synthetic regulatory nucleic acid molecule is active in cells of at least two different bacilli species.

11. The method of claim 10, wherein the synthetic regulatory nucleic acid molecule is active in cells of at least one bacilli specie.

12. The method of claim 8, wherein the synthetic regulatory nucleic acid molecule is active in cells of least one of the group consisting of Bacillus subtilis, Bacillus licheniformis and Bacillus pumilus.

13. The method of claim 12, wherein the synthetic regulatory nucleic acid molecule is active in cells of Bacillus licheniformis.

14. The method of claim 1 wherein the starting regulatory nucleic acid molecule conferring constitutive expression in a bacterial cell is selected from the group consisting of
a. SEQ ID NO: 28 and 29,
b. a nucleic acid molecule comprising at least 20 consecutive base pairs identical to 20 consecutive base pairs of a sequence described by SEQ ID NOs: 28 or 29,
c. a nucleic acid molecule having an identity of at least 90% over the entire length of a sequence described by SEQ ID NO: 28 or 29,
d. a nucleic acid molecule hybridizing under high stringent conditions with a nucleic acid molecule of at least 20 consecutive base pairs of a nucleic acid molecule described by SEQ ID NO: 28 or 29, and
e. a complement of any of the nucleic acid molecules as defined in a) to d).

15. A synthetic regulatory nucleic acid molecule wherein the regulatory nucleic acid molecule is selected from the group consisting of
f. a nucleic acid molecule having a sequence of SEQ ID NO 35, 36, 37, 38, 39, 40, 42, 43, 45, 46 or 47,
g. a nucleic acid molecule comprising at least 20 consecutive base pairs identical to 20 consecutive base pairs of a sequence described by SEQ ID NO: 35, 36, 37, 38, 39, 40, 42, 43, 45, 46 or 47,
h. a nucleic acid molecule having an identity of at least 90% over the entire length to a sequence described by SEQ ID NO: 35, 36, 37, 38, 39, 40, 42, 43, 45, 46 or 47,
i. a nucleic acid molecule hybridizing under high stringent conditions with a nucleic acid molecule of at least 20 consecutive base pairs of a nucleic acid molecule described by SEQ ID NO: 35, 36, 37, 38, 39, 40, 42, 43, 45, 46 or 47, and
j. a complement of any of the nucleic acid molecules as defined in a) to d),
wherein the sequences as defined in b) to e) are distinct from SEQ ID NO: 28 or 29.

16. A synthetic regulatory nucleic acid molecule comprising a nucleic acid molecule produced as defined in claim 1.

17. An expression construct comprising a synthetic regulatory nucleic acid molecule of claim 15.

18. A vector comprising a regulatory nucleic acid molecule of claim 15.

19. A microorganism comprising a regulatory nucleic acid molecule of claim 15.

20. An expression construct comprising a synthetic regulatory nucleic acid molecule of claim 16.

21. A vector comprising a regulatory nucleic acid molecule of claim 16.

22. A microorganism comprising a regulatory nucleic acid molecule of claim 16.


SEQUENCE LISTING (104 sequences)

SEQ ID NO: 1 - DNA, 32 bp, Artificial Sequence
  Oligonucleotide PCR-amplification of pE194
tatatacagctggattcacaaaaaataggcac

SEQ ID NO: 2 - DNA, 33 bp, Artificial Sequence
  Oligonucleotide PCR-amplification of pE194
tatatacagctggattatgtcttttgcgcagtc

SEQ ID NO: 3 - DNA, 30 bp, Artificial Sequence
  Oligonucleotide PCR-amplification of functional mRFP cassette
tatatggatccgtaatcagggtatcgaggc

SEQ ID NO: 4 - DNA, 31 bp, Artificial Sequence
  Oligonucleotide PCR-amplification of functional mRFP cassette
tatatggatccctcattaggcgggctactaa

SEQ ID NO: 5 - DNA, 1345 bp, Artificial Sequence
  functional fragment T2A mRFP cassette
ggaatagatctggccaacgaggcctcgaggatccgatatcatgcatggcgcgccaccctgagacgaccctgatgcaggtgaccctgagaccttcgcgcccagctgtctagggcggcggatttgtcctactcaggagagcgttcaccgacaaacaacagataaaacgaaaggcccagtctttcgactgagcctttcgttttatttgatgcctttaattaagtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagtgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttggtctcactcacacctgctcgtctcactcaatttaaatggcggccgcggatcctcgacgggccaataaggccagatctggatt

SEQ ID NO: 6 - DNA, 1012 bp, Artificial Sequence
  homology regions Fusion of 5- and 3-prime regions of amyB gene with flanking XmaI sites
cccgggataatgccgtcgcactggccgatattgagagatttccttgtgacaagctgcaaagcataatgatgacggtccagctcgcggctgattcccgttaacagattcatataataaggttctgttgtatccatttcttccagtatgagcagcttgacgacctgtgttctgttttgaacgagcgctcttgctgcatagttcggtatataattgagctccttcattgcggaatgaacaagctttttcaattcatccgtcacagtctcaggatgattgatcacccgcgataccgtcattttcgacacatttgctttctttgctacatcagataacgttgccatttcatccccgccttacctatgcgattcaaactgtcagcaagtccttcctgagggctgatgacactttgttaaaattaattataaaatgtaatcaaagaaatttataagacgggcaaaataaaaaaacggatttccttcaggaaatccgtcctctctgctcttctagattctcctcccctttcaatgtgaaacatatgatattgtataaatattccgaatttttaacaaataccattttccctatattttcttccaaaagaaaagcgccgatatggcgctttctactcatttattcaatagcctctctgcttcttcacttcttcaagctgagatacagttaccaattgatagccttttgctttcagctttttaataatctcttcagcagcatctgcggacgttgcataaatatcgtgcattaagacgatttttccgtctcccgcatggctcatgacatgattgacaatcttttgcttatttttgtacttccaatcttccggatcaacatcccacaatgaaaccttcagattggaaagcgagcggacggaatcattgatcccgccgtatggaggacgcaagtgtacaggcaggtgtccgctgattttttcgatcatttcttgcgtgtcgttaatctcctgatacgctttttcgtttgacagccttgtcccggg

SEQ ID NO: 7 - DNA, 24 bp, Artificial Sequence
  Oligonucleotide Protospacer target sequence for amyB gene with 4nt 5-prime extension
tacgtcgcagcagaaattaagaga

SEQ ID NO: 8 - DNA, 24 bp, Artificial Sequence
  Oligonucleotide Protospacer target sequence for amyB gene with 4nt 5-prime extension
aaactctcttaatttctgctgcga

SEQ ID NO: 9 - DNA, 20 bp, Artificial Sequence
  Oligonucleotide PCR-amplification of amyB genomic region
ttgcccgaatacaacgacag

SEQ ID NO: 10 - DNA, 21 bp, Artificial Sequence
  Oligonucleotide PCR-amplification of amyB genomic region
caacaacaggctgtctgacgg

SEQ ID NO: 11 - DNA, 2143 bp, Bacillus licheniforms
  P300 DSM641 RMS RMS region and flanking region
aacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacattatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttagg

SEQ ID NO: 12 - DNA, 1146 bp, Bacillus licheniforms
  cds Restrictase P300 DSM641
ttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacat

SEQ ID NO: 13 - DNA, 1022 bp, Artificial Sequence
  homology regions Fusion of 5- and 3-prime regions of restrictase gene with flanking BsaI sites
ggtctcgacccaacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttaggctcaggagacc

SEQ ID NO: 14 - DNA, 21 bp, Artificial Sequence
  Oligonucleotide PCR-amplification of restrictase genomic region
gacaatccccttttactgacc

SEQ ID NO: 15 - DNA, 24 bp, Artificial Sequence
  Oligonucleotide PCR-amplification of restrictase genomic region
ctatttcatttgcccagacaatcc

SEQ ID NO: 16 - DNA, 1363 bp, Artificial Sequence
  homology regions Fusion of 5- and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 17 - DNA, 20 bp, Artificial Sequence
  Oligonucleotide PCR-amplification of pga genomic region
aaagccttctcctctctatt

SEQ ID NO: 18 - DNA, 20 bp, Artificial Sequence
  Oligonucleotide PCR-amplification of pga genomic region
ttcttgaaaaagacaaggtc

SEQ ID NO: 19 - DNA, 1027 bp, Artificial Sequence
  homology regions Fusion of 5- and 3-prime regions of the aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 20 - DNA, 21 bp, Artificial Sequence
  Oligonucleotide PCR-amplification of aprE genomic region
ccggttgtcattgatccttta

SEQ ID NO: 21 - DNA, 21 bp, Artificial Sequence
  Oligonucleotide PCR-amplification of aprE genomic region
atcctcctgcaaaaaccgtat

SEQ ID NO: 22 - DNA, 41 bp, Artificial Sequence
  Oligonucleotide PCR-amplification of pBW742
caaatttacaaaagcgactcgtgagttttcgttccactgag

SEQ ID NO: 23 - DNA, 36 bp, Artificial Sequence
  Oligonucleotide PCR-amplification of pBW742
gaacgttgctctagagttaagggattttggtcatgg

SEQ ID NO: 24 - DNA, 38 bp, Artificial Sequence
  Oligonucleotide PCR-amplification of terminator region of pMutin2
gtggaacgaaaactcacgagtcgcttttgtaaatttgg

SEQ ID NO: 25 - DNA, 40 bp, Artificial Sequence
  Oligonucleotide PCR-amplification of terminator region of pMutin2
catgaccaaaatcccttaactctagagcaacgttcttgcc

SEQ ID NO: 26 - DNA, 22 bp, Artificial Sequence
  functional fragment region of terminator region of pMutin2
ggggatctctgcagtgagatct

SEQ ID NO: 27 - DNA, 24 bp, Artificial Sequence
  Sequence of terminator region in pCC009
ggggatctctgcagtcgggaagat

SEQ ID NO: 28 - DNA, 128 bp, Artificial Sequence
  promoter variant
aattttgtcaaaataattttattgacaacgtcttattaacgttgataccggttaaattttatttgacaaaaatgggctcgtgttggagaataaatgtggagaaagattaactaataaggaggacaaac

SEQ ID NO: 29 - DNA, 128 bp, Artificial Sequence
  promoter variant
aattttgtcaaaataattttattgacaacgtcttattaacgttgataccggttaaattttatttgacaaaaatgggctcgtgttgaagaataaatgtggagaaagattaactaataaggaggacaaac

SEQ ID NO: 30 - DNA, 101 bp, Artificial Sequence
  Oligonucleotide
aacacgagcccatttttgtcaaataaaatttaaccggtatcaacgttaataagacgttgtcaataaaattattttgacaaaattttaataatccaaatgag

SEQ ID NO: 31 - DNA, 87 bp, Artificial Sequence
  Oligonucleotide
ctattgagtatttcttatccatgtttgtcctccttattagttaatctttctccacatttattctccaacacgagcccatttttgtca

SEQ ID NO: 32 - DNA, 48 bp, Artificial Sequence
  Oligonucleotide
gattaactaataaggaggacaaacatggataagaaatactcaataggc

SEQ ID NO: 33 - DNA, 87 bp, Artificial Sequence
  Oligonucleotide
ctattgagtatttcttatccatgtttgtcctccttattagttaatctttctccacatttattcttcaacacgagcccatttttgtca

SEQ ID NO: 34 - DNA, 127 bp, Artificial Sequence
  promoter variant
aattttgtcaaaataattttattgacaacgtcttattaacgttgataccggttaaattttatttgacaaaatgggctcgtgttggagaataaatgtggagaaagattaactaataaggaggacaaac

SEQ ID NO: 35 - DNA, 126 bp, Artificial Sequence
  promoter variant
aattttgtcaaaataattttattgacaacgtcttattaacgttgataccggttaaattttatttgacaaaaatgggctcgtgttggagaaaatgtggagaaagattaactaataaggaggacaaac

SEQ ID NO: 36 - DNA, 127 bp, Artificial Sequence
  promoter variant
aattttgtcaaaataattttattgacaacgtcttattaacgttgataccggttaaattttatttgacaaaaatgggctcgtgttgggaataaatgtggagaaagattaactaataaggaggacaaac

SEQ ID NO: 37 - DNA, 129 bp, Artificial Sequence
  promoter variant
aattttgtcaaaataattttattgacaacgtcttattaacgttgataccggttaaattttatttgaacaaaaatgggctcgtgttggagaataaatgtggagaaagattaactaataaggaggacaaac

SEQ ID NO: 38 - DNA, 127 bp, Artificial Sequence
  promoter variant
aattttgtcaaaataattttattgacaacgtcttattaacgttgataccggttaaattttatttgacaaaaatgggctcgtgttgagaataaatgtggagaaagattaactaataaggaggacaaac

SEQ ID NO: 39 - DNA, 127 bp, Artificial Sequence
  promoter variant
aattttgtcaaaataattttattgacaacgtcttattaacgttgataccggttaaattttatttgacaaaaatgggctcgtgttggaaataaatgtggagaaagattaactaataaggaggacaaac

SEQ ID NO: 40 - DNA, 127 bp, Artificial Sequence
  promoter variant
aattttgtcaaaataattttattgacaacgtcttattaacgttgataccggttaaattttatttgacaaaaagggctcgtgttggagaataaatgtggagaaagattaactaataaggaggacaaac

SEQ ID NO: 41 - DNA, 127 bp, Artificial Sequence
  promoter variant
aattttgtcaaaataattttattgacaacgtcttattaacgttgataccggttaaattttatttgacaaaaatgggctcgtgttgaagaaaaatgtggagaaagattaactaataaggaggacaaac

SEQ ID NO: 42 - DNA, 130 bp, Artificial Sequence
  promoter variant
aattttgtcaaaataattttattgacaacgtcttattaaccgttgataccggttaaattttatttgacaaaaatgggctcgtgttgaagaataaatgtggagaaagattaactaataagggaggacaaac

SEQ ID NO: 43 - DNA, 127 bp, Artificial Sequence
  promoter variant
aattttgtcaaaataattttattgacaacgtcttattaacgttgataccggttaaattttatttgacaaaaatgggctcgtgttgaagaataaatgtggaaaagattaactaataaggaggacaaac

SEQ ID NO: 44 - DNA, 128 bp, Artificial Sequence
  promoter variant
aattttgtcaaaataattttattgacaacgtcttattaacgttgatacccggttaaattttatttgacaaaaatgggctcgtgtgaagaataaatgtggagaaagattaactaataaggaggacaaac

SEQ ID NO: 45 - DNA, 127 bp, Artificial Sequence; identical to SEQ ID NO: 38
  promoter variant

SEQ ID NO: 46 - DNA, 127 bp, Artificial Sequence
  promoter variant
aattttgtcaaaataattttattgacaacgtcttattaacgttgataccggttaaattttatttgacaaaatgggctcgtgttgaagaataaatgtggagaaagattaactaataaggaggacaaac

SEQ ID NO: 47 - DNA, 129 bp, Artificial Sequence
  promoter variant
aattttgtcaaaataattttattgacaacgtcttattaacgttgataccggttaaattttattttgacaaaaatgggctcgtgttgaagaataaatgtggagaaagattaactaataaggaggacaaac

SEQ ID NO: 48 - DNA, 40 bp, Artificial Sequence
  Oligonucleotide
gaagttttagatgccactcttatccatcaatccatcactg

SEQ ID NO: 49 - DNA, 41 bp, Artificial Sequence
  Oligonucleotide
catctcaaatttcgcatttattccaatttcctttttgcgtg

SEQ ID NO: 50 - DNA, 40 bp, Artificial Sequence
  Oligonucleotide
cacacgcaaaaaggaaattggaataaatgcgaaatttgag

SEQ ID NO: 51 - DNA, 40 bp, Artificial Sequence
  Oligonucleotide
gaccagtgatggattgatggataagagtggcatctaaaac

SEQ ID NO: 52 - DNA, 20 bp, Artificial Sequence
  Oligonucleotide
caagctatgcttgctcaagc

SEQ ID NO: 53 - DNA, 50 bp, Artificial Sequence
  Oligonucleotide
ctgttggttcgcttgagcaagcatagcttggacggttcagcgtgttaagc

SEQ ID NO: 54 - DNA, 37 bp, Artificial Sequence
  Oligonucleotide
ggtggtggtctctaccccgatcgaagagccattcgag

SEQ ID NO: 55 - DNA, 37 bp, Artificial Sequence
  Oligonucleotide
ggtggtggtctcttgagcttcctctgtccgattgtcc

SEQ ID NO: 56 - DNA, 24 bp, Artificial Sequence
  Oligonucleotide
tacggcaatctctgaaaaaatgag

SEQ ID NO: 57 - DNA, 24 bp, Artificial Sequence
  Oligonucleotide
aaacctcattttttcagagattgc

SEQ ID NO: 58 - DNA, 814 bp, Artificial Sequence
  homology regions Homology region of the mutated degU gene with flanking BsaI sites
ggtctcgacccgatttgggattgataccgacgctcagaaaatacttgaacacgatcgaagattatcatggaaaagcaaagattcatttccaatgcatcggagaatccgaagaaagaagaatagcaccgcggtttgaggttgcactattccggcttgcacaggaagcggtgacaaacgccttaaaacactccgaatcaactgaaattcatgttaaagtagaagtgacaaaagattttgtgacgctgattatcaaagacaatggaaacggctttgacttaaaagaagtaaaaggcaagaagaacaaatctttcggtctgctaggtatgaaagaaagagtcgatttgctcgaaggctcaatgacaatcgattcgaaaataggtcttgggacatttatattgattaaagttccactgtctttgtaaagataattgtaaaatagagacaaaagacatattgaccataaaagcggtgtgtttaacaatgagaatggggaggcgtagcttgtgactaaagtaaatattgtaattattgacgatctgcagttattccgtgaaggtgtcaaacggattttggatttcgagcctacctttgaagtagtggccgaaggagacgacggagatgaagcggctcgcattgtcgagcactaccatcctgatgttgttatcatggatattaatatgccgaatgtgaacggagtagaagcgacaaaacaactggtcgacttgtatccggaatcaaaggttattattttatccatccatgatgacgaaaactatgttacacatgcattaaaaacaggagccctcaggagacc

SEQ ID NO: 59 - DNA, 24 bp, Artificial Sequence
  Oligonucleotide
tacgggatttcgaacctacctttg

SEQ ID NO: 60 - DNA, 24 bp, Artificial Sequence
  Oligonucleotide
aaaccaaaggtaggttcgaaatcc

SEQ ID NO: 61 - DNA, 39 bp, Artificial Sequence
  Oligonucleotide
ggttgcggtctcatacgtgaagatcaggctatcactggt

SEQ ID NO: 62 - DNA, 36 bp, Artificial Sequence
  Oligonucleotide
caacgggtctcttgagttggcaggccgctgaatttc

SEQ ID NO: 63 - DNA, 1435 bp, Artificial Sequence
  sgRNA and homology regions protospacer aprE-sgRNA, homology region of the aprE gene with flanking BsaI sites
ggttgcggtctcatacggaaacaaacccataccaggagttttagagctagaaatagcaagttaaaataaggctagtccgttatcaacttgaaaaagtggcaccgagtcggtgctttttactccatctggatttgttcagaacgctcggttgccgccgggcgttttttatctaaagcttaggcccagtcgaaagactgggcctttttaatacgactcactatagggtcgacggccaacgaggcccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagataatagtaaaaagaagcaggttcctccatacctgcttctttttatttgtcagcatcctgatgttccggcgcattctcttctttctccgcatgttgaatccgttccatgatcgacggatggctgcctctgaaaatcttcacaagcaccggaggatcaacctggctcagccccgtcacggccaaatcctgaaacgttttaacagcggcttctctgttctctgtcaactcgatcccatactggtcagccttattctcctgataacgcgagacagcattagaaaaaggcgtaaccgcaaagctcaaaacagaaaacaaaagcaataacagcggaagtgccgcaagatcatgccgcccttctaaatgaaacatgctgcgggttaggcgaaccgtccgcttgtaaagcttatcaatgacataaaatccggcgagcgacacgagcaaatagccagccagaccgatgtaaacgtgcttcatgacataatggcccatttcgtggcccataataaacagaatttctgaatcgtcaagtttgttcagcgtcgtatcccacaatacaatccgtttattggccccaattctcaagagacccgttg

SEQ ID NO: 64 - DNA, 20 bp, Artificial Sequence
  Protospacer protospacer target sequence for aprE gene
gaaacaaacccataccagga

SEQ ID NO: 65 - DNA, 1241 bp, Artificial Sequence
  sgRNA and homology regions protospacer vpr-sgRNA, homology region of the vpr gene with flanking BsaI sites
ggttgcggtctcatacgatcaggaacggaacgagtcggttttagagctagaaatagcaagttaaaataaggctagtccgttatcaacttgaaaaagtggcaccgagtcggtgctttttactccatctggatttgttcagaacgctcggttgccgccgggcgttttttatctaaactagtacagtcgatacggatccactagtctatctcttctcttttttccgaaaagccgcctgcctgataagcatcggcagcctgatatgtaccgccggcgaacgccgtcaccttcatctgattttctctgacaggcatcaccgcattcagcagcacggctgtccataattttaaaaatgatatcaaacctttcataccgatccctccagtttcgttttgataaaactagcaactctattaaactttcttgctctatcttatcccagcaaaatgaaaatgtttgtcacaatgtgtgtgcaaaatgattctagtttttagaagttttgttgaaaactgaaggaatcgcatgattcagcggatacaaaccatgaatgtaacttactcacagcttatcctaaggataaacacatattacccacaggatatatccacatatccacatacttattcaatatttagtataagaacgtatattccctacaatatctatacacaagtttattcacttatacacagtaaattgtgcataaatctaatgacaagccttgttgagaaccactcaacaaggcttttttatgttaaaatacggataatgcgttcaggagaagctccccttctcttcaaaacgtgaaaaaagcaatcggaggacatcgtgtatatgctttcttttatcgtattattcggcttatccttcattattgtctgctttatatttttcacgactttgtacttcgccgtcaacctgcagaagcgcgagcccaagccttttcaaaaagctgcggagcaaaccgtcgataccatcatcctcattccgctcagctggctgtttaccgctttatacatatgcattctgtttattcttttcccaatccgccattttctcgatttttttcagcaaaaacgctaaattgactgatgaaacgcttcggccagcagccggtatgaatccaatctgtcttgaaaatcgtgggtgatcgtcaccgccatgatttcgtccgttccgtaagcgccggccagttcaagcagctgttcctagctcgagccatggctcactcaagagacccgttg

SEQ ID NO: 66 - DNA, 20 bp, Artificial Sequence
  Protospacer protospacer target sequence for vpr gene
atcaggaacggaacgagtcg

SEQ ID NO: 67 - DNA, 20 bp, Artificial Sequence
  Protospacer protospacer target sequence for vpr gene
gcttccgtataatgagtatt

SEQ ID NO: 68 - DNA, 20 bp, Artificial Sequence
  Protospacer protospacer target sequence for vpr gene
cacgatccgaaaaacccgta

SEQ ID NO: 69 - DNA, 1338 bp, Artificial Sequence
  sgRNA and homology regions protospacer epr-sgRNA, homology region of the epr gene with flanking BsaI sites
ggttgcggtctcatacgattccggtccagcgcttttagttttagagctagaaatagcaagttaaaataaggctagtccgttatcaacttgaaaaagtggcaccgagtcggtgctttttactccatctggatttgttcagaacgctcggttgccgccgggcgttttttatctaaactagtaggcccagtcgatacgtaaagactgggcctttttaatacgactcactatagggtcgacggccaacgaggcctcgaggatccgatatcatgcatggcgcgccaccctgagacgaccctgatgcaggtgacccggatccactagtccgatttgacatcgtgctgtttaaaggacctgacaaagatatattcattaaaagggtgatcgggcttccgggcgaaaccctcaggtatgaagatgatcagctgtatatcaacgaagaaaagatcaaagagccttatctggacgacttaaaggccgtcaccgccggaggggacttgacaggggattttacactgcaggaagtgaccggagaggagaaggtgcctgaaaacgagtacttcgtcctcggggacaaccggatccacagctttgacagccgccatttcggctttgtttcagaacgggacatcgtcgggattgtgacggaaagaattgataagaagtgattggagagtacgggggagagtaagcggccgaccaaggaatacgattacgcaaatgacgagcccgaaatgtcaattagtacaacagcatcaataatgacgcatttgctaaatatgaaaattaaaaggcccggatgattccgggcttttttccgtactaagcggcgttcgctatatatatcggaggatttttgaattttcaaagagaaagaaagcttatcttaaggtcgcttgtcatgcacctttagtttttaaaacgttaaaaacactgttatatcaacatttgtgaagcttcctgtttattcgggaagttaaattgggtactccaagttagttttaaaaaagagtcataaggccagcttctatcgatgaatcatttttaagcgacgccttttgtctaaaatgtataatgttacttttgtttttgttgaaagtgaacaatgttattgactggcttacaacctacaatttaattaaataaaaaatagattaaaagaagggagcgttctcataccgtggaaaaaacaattaaacatgaccccaattattacaaaaagataattattgcattgtgtttagggtgggtcgctatttggatttatcgtacaatacttacgccaatatatccgcagattcaagaatcattagggaatattagtgctcaagagacccgttg

SEQ ID NO: 70 - DNA, 20 bp, Artificial Sequence
  Protospacer protospacer target sequence for epr gene
attccggtccagcgctttta

SEQ ID NO: 71 - DNA, 20 bp, Artificial Sequence
  Protospacer protospacer target sequence for epr gene
cgctttttcagctttggcaa

SEQ ID NO: 72 - DNA, 20 bp, Artificial Sequence
  Protospacer protospacer target sequence for epr gene
gcaaacatgccggtgacgtg

SEQ ID NO: 73 - DNA, 865 bp, Artificial Sequence
  functional fragment T2A lacZ cassette
gcgatgttaaggaccgtctcatctcacctgcaaggtctcatctcttttactccatctggatttgttcagaacgctcggttgccgccgggcgttttttatctaaaactagtgtcgagggtcttcggtaccgcgatttacatatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgccgcgtttataatgaagaccctagcaggcatcaaataaaacgaaaggctcagtcgaaagactgggcctttcgttttatctgttgtttgtcggtgaacgctctcctgagtaggacaaatccgccgccctagacagctgtcgctgagacgatcgctgagacctcgcaagttctcgccatcgcaggtgaaatctagatgtattcgc

SEQ ID NO: 74 - DNA, 37 bp, Artificial Sequence
  Oligonucleotide PCR-amplification of promoter PaprE
tatatgaagaccttcgactcgggacctctttccctcg

SEQ ID NO: 75 - DNA, 41 bp, Artificial Sequence
  Oligonucleotide PCR-amplification of promoter PaprE
tatagaagacttatatctcactctcctcctctttattcaga

SEQ ID NO: 76 - DNA, 747 bp, Artificial Sequence
  functional fragment GFPmut2 AF302837 with flanking BpiI restriction sites
attagaagacctatatgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttcgcgtatggtcttcaatgctttgcgagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataatagcttgtcttcatta

SEQ ID NO: 77 - DNA, 33 bp, Artificial Sequence
  Oligonucleotide PCR-amplification of amyB genomic region
tataggtctcaacccataatgccgtcgcactgg

SEQ ID NO: 78 - DNA, 35 bp, Artificial Sequence
  Oligonucleotide PCR-amplification of amyB genomic region
ccttggtctcgtcgctagaagagcagagaggacgg

SEQ ID NO: 79 - DNA, 39 bp, Artificial Sequence
  Oligonucleotide PCR-amplification of amyB genomic region
ttgagaggtctcagagacattttccctatattttcttcc

SEQ ID NO: 80 - DNA, 35 bp, Artificial Sequence
  Oligonucleotide PCR-amplification of amyB genomic region
gtgaggtctcatgagaccatccgttattgacaagg

SEQ ID NO: 81 - DNA, 20 bp, Artificial Sequence
  Protospacer protospacer target sequence for sigE gene
cggtgaggaaaaaacccaaa

SEQ ID NO: 82 - DNA, 1338 bp, Artificial Sequence
  sgRNA and homology regions protospacer epr-sgRNA, homology region of the sigE gene with flanking BsaI sites
ggttgcggtctcatacgcggtgaggaaaaaacccaaagttttagagctagaaatagcaagttaaaataaggctagtccgttatcaacttgaaaaagtggcaccgagtcggtgctttttactccatctggatttgttcagaacgctcggttgccgccgggcgttttttatctaaactagtaggcccagtcgatacgtaaagactgggcctttttaatacgactcactatagggtcgacggccaacgaggcctcgaggatccgatatcatgcatggcgcgccaccctgagacgaccctgatgcaggtgacccggatccactagtaagacagtatgatgaacaagtgcttgtggaactacacattcacggagagacaattcgtttaaagggactcgtcgattctggcaaccagctgtatgatcctatgaccaaaacaccggtcatgatcgtccaggccgaccatctaacggccatttgcggagaatcgtttatagaccttatgaaacagtctcatcctgttgaagtcatgcaaaagatcgatgatcaatttcctcttcttgatcgattaagacttgttccatatcgagcagtcggtcatgatcacggttttctactatgcctaaaaccagatacagttgtcatttattcaaagacgcatatgattcagccagctaagtgttttgtaggattgagtctgagcggcttatcggcagatcaggaatttcaatccatcattcatccagatatgttagacgggaaaatcatccagggggtgtcgtagtttttggtgatgtcttttatcttacgaggtcaacttgacattttgaaaaatttttttgaaaagctctgtccctgtactgtcaaaggaaacaaccttttttctcatgattctcgtcatcgctcgtgcatatttttccaacccaaggagatactgaactttgtacaacagctcctgtagggagggaaaaaagtgtccagaaataaagtggaaatctgcggagtcgacacctccaagctgcctgttctgaaaaacgacgagatgagaaaattgttcagacagctgcaagatgagggtgacgatacagcaagagaaaagctagtcaatggcaatttacggttggttttaagtgtgattcagcgtttcaacaacagaggcgagtatgtcgatgatctctttcaagtaggctgtatcggattaatgaaatcaattgataattttgatttaagccacaatgtcagattttcaacttatgcggttcctatgatcataggagaaatccgtcgatacttgcgtgataataatccgattcgggtgtctcgctcactcaagagacccgttg

SEQ ID NO: 83 - DNA, 20 bp, Artificial Sequence
  Protospacer protospacer target sequence for sigF gene
tgtcgtcctcgctcaagaag

SEQ ID NO: 84 - DNA, 1338 bp, Artificial Sequence
  sgRNA and homology regions protospacer epr-sgRNA, homology region of the sigF gene with flanking BsaI sites
ggttgcggtctcatacgtgtcgtcctcgctcaagaaggttttagagctagaaatagcaagttaaaataaggctagtccgttatcaacttgaaaaagtggcaccgagtcggtgctttttactccatctggatttgttcagaacgctcggttgccgccgggcgttttttatctaaactagtaggcccagtcgatacgtaaagactgggcctttttaatacgactcactatagggtcgacggccaacgaggcctcgaggatccgatatcatgcatggcgcgccaccctgagacgaccctgatgcaggtgacccggatccactagtaaattatccgtatggagccttcagagcaaacggcattgcaaacattgggggtggcatcatgaggaatgaaatgaacctgaccttctctgccttaagtcaaaatgaatcctttgcgagggtgacagtcgcggcgtttatcgcccagcttgatccgacattagatgaattaactgaaatcaaaaccgttgtgtcagaggcggtgacaaactccattatccatggctatgatgggaatccagatggcaaggtgcatattgaagtcacacttgatgatcatgttgtgtacctgaccatccgtgacgaaggaatgggtattacagatcttgaggaagcaagacagccgcttttcacgacaaaaccagacttagaacgctctggcatgggctttaccattatggagaattttatggatgatgtcatgatagactcatctccagaaatgggcacaaccatccgtttaacaaagcatctatcaaaaagcaaagcgctttgtaattaaatagccaattcggctggctttttttgtgtggtaattaccggtaaatgaagttctctcggtatgagaaccatttttcaccacatactatttttaacccatcgtataggaagtgacttggatggacggacaaatctttcttcggctgcgccatcgcattaagaccggtaatgatcagctcatttatttagaagatatcgcccaaatcactggtgatgagttggctgtgcaaaagcttagcaagatgccgatatatcatgtcagtaaaaaggatcgtcacattgccgttcttgatatcatgcatgtggtcaaaacgatcaaaaaaacatggccaaccatcgacattcaaactgtcggaggcgctgaagccattgttgaaattgatacaggcaaacgccagctttctcccgtattatttgtgttcgtgtggcttttattatttgtcggagcggcgcttgccattatgaatttccacgaggatgtcagtatgcggctcgtccatatctcaagagacccgttg

SEQ ID NO: 85 - DNA, 20 bp, Artificial Sequence
  Protospacer protospacer target sequence for spoIIE gene
cttgtagctgaacagctgat

SEQ ID NO: 86 - DNA, 1338 bp, Artificial Sequence
  sgRNA and homology regions protospacer epr-sgRNA, homology region of the spoIIE gene with flanking BsaI sites
ggttgcggtctcatacgcttgtagctgaacagctgatgttttagagctagaaatagcaagttaaaataaggctagtccgttatcaacttgaaaaagtggcaccgagtcggtgctttttactccatctggatttgttcagaacgctcggttgccgccgggcgttttttatctaaactagtaggcccagtcgatacgtaaagactgggcctttttaatacgactcactatagggtcgacggccaacgaggcctcgaggatccgatatcatgcatggcgcgccaccctgagacgaccctgatgcaggtgacccggatccactagtcctgctgtccgcaggtgctttttttcttgacccacacgacattttttgagatttcgtcatttaatttaaaacttcctattgacggacaagcgattcctttgtattatagatcttgtgcttcttagcgcatttttattatggcggtgtagctcagctggctagagcgtacggttcatacccgtgaggtcgggggttcgatcccctccgccgctatccttttgattagaacataaaagcaaggcccgttggtcaagcggttaagacaccgccctttcacggcggtaacacgggttcgaatcccgtacgggtcatcttcgaaaacagctttctttaggaaagctgtttttttgtgtcttcataaaattctgatgaaagacatcgactttcaagaaagtatgcctctttgacgaataaagcgtcgaacgttttatggaaacgacaacttcttttgacaaaatttctttttcaccttcgctataatgacaagcaacgaatatcagtgaaatatcgtataatatgaatttcttctggcgatgatggggatataaagcattcagtacgatcccaggaggaatgaagatgcgaaaaggtcacgtaaaccaaatcttattgattacagatggctgctcaaatcacggggaagatccacttgcgattgcctcattggcaaaggaacaagggattacagtcaatgttattggcattatggaggaaaacagacacgaccatgaagcaatgaaagaagttgaagggattgctctcgcaggtggaggcatccatcaagttgtctacgtccagcagttatctcaaaccgtacaaatggttacaaaaaaagcgatgacacaaaccttgcaaggtgttgtgaataaagaattgcagcaaatacttggcaaggacactgaaattgaagagctgccacctgataaacgcggggaagtgatggaagtagtcgatgagttaggagagacggttcatcttcaagtgcttgtgcttgttgatactcaagagacccgttg

SEQ ID NO: 87 - DNA, 20 bp, Artificial Sequence
  Oligonucleotide PCR-amplification of hag genomic region
tgatcttgatgaaacgacgg

SEQ ID NO: 88 - DNA, 22 bp, Artificial Sequence
  Oligonucleotide PCR-amplification of hag genomic region
taatcctgatattctgatcgcc

SEQ ID NO: 89 - DNA, 19 bp, Artificial Sequence
  Oligonucleotide PCR-amplification of degU genomic region
atgatttaaggccgatggc

SEQ ID NO: 90 - DNA, 21 bp, Artificial Sequence
  Oligonucleotide PCR-amplification of degU genomic region
atccgccttcagctactactt

SEQ ID NO: 91 - DNA, 26 bp, Artificial Sequence
  Oligonucleotide PCR-amplification of amyE genomic region
ggtcatgaataatctgcgtaatagac

SEQ ID NO: 92 - DNA, 26 bp, Artificial Sequence
  Oligonucleotide PCR-amplification of amyE genomic region
gcgtgtacgttttgaggcgctgcgcc

SEQ ID NO: 93 - DNA, 26 bp, Artificial Sequence
  Oligonucleotide PCR-amplification of aprE genomic region B. subtilis
gagctggcagatgaagccaatattcc

SEQ ID NO: 94 - DNA, 27 bp, Artificial Sequence
  Oligonucleotide PCR-amplification of aprE genomic region B. subtilis
gtacgcgcatgaggaacgacaaataag

SEQ ID NO: 95 - DNA, 24 bp, Artificial Sequence
  Oligonucleotide PCR-amplification of vpr genomic region
ctgtcacccacttcccattatgag

SEQ ID NO: 96 - DNA, 24 bp, Artificial Sequence
  Oligonucleotide PCR-amplification of vpr genomic region
gtgaccgaaggctttccatcattg

SEQ ID NO: 97 - DNA, 24 bp, Artificial Sequence
  Oligonucleotide PCR-amplification of epr genomic region
cttgtcatcgtcgtcgggattcag

SEQ ID NO: 98 - DNA, 24 bp, Artificial Sequence
  Oligonucleotide PCR-amplification of epr genomic region
gtgccaatcacaaatgtagccagc

SEQ ID NO: 99 - DNA, 25 bp, Artificial Sequence
  Oligonucleotide PCR-amplification of sigE genomic region
gaggaggcatgatcggagttcattc

SEQ ID NO: 100 - DNA, 25 bp, Artificial Sequence
  Oligonucleotide PCR-amplification of sigE genomic region
ctcctccgtcattatagatcggttc

SEQ ID NO: 101 - DNA, 25 bp, Artificial Sequence
  Oligonucleotide PCR-amplification of sigF genomic region
gtgacgaattatttggaaacagagg

SEQ ID NO: 102 - DNA, 25 bp, Artificial Sequence
  Oligonucleotide PCR-amplification of sigF genomic region
cgacataatgatcgagatcgagctg

SEQ ID NO: 103 - DNA, 25 bp, Artificial Sequence
  Oligonucleotide PCR-amplification of spoIIE genomic region
ctcaacaacaacaatcaagaccgag

SEQ ID NO: 104 - DNA, 25 bp, Artificial Sequence
  Oligonucleotide PCR-amplification of spoIIE genomic region
gatagtgaatcgaatcgaggcgtcc